(19) United States
(12) Patent Application Publication
(10) Pub. No.: US 2024/0374538 A1
(43) Pub. Date: Nov. 14, 2024

(54) COMPOSITIONS COMPRISING NATURAL PRODUCTS FOR IMPROVING CARBOHYDRATE AND LIPID METABOLISM

(71) Applicant: GROUPE NATURE PHARMA, Pointe Claire (CA)

(72) Inventor: Alexandro ZARRK, Quebec (CA)

(21) Appl. No.: 18/659,926

(22) Filed: May 9, 2024

Related U.S. Application Data
(60) Provisional application No. 63/501,521, filed on May 11, 2023.
(60) Provisional application No. 63/617,955, filed on Jan. 5, 2024.

(51) Int. Cl. A61K 31/122; A61K 9/48; A61K 31/51; A61K 33/06; A61K 33/24; A61K 33/30; A61K 33/34; A61K 36/258; A61K 36/42; A61K 36/48; A61K 36/54; A61K 36/9068; A61P 3/10

(52) U.S. Cl. CPC A61K 31/122; A61K 9/48; A61K 31/51; A61K 33/06; A61K 33/24; A61K 33/30; A61K 33/34; A61K 36/258; A61K 36/42; A61K 36/48; A61K 36/54; A61K 36/9068; A61P 3/10

(57) ABSTRACT

The present application relates to compositions, nutritional supplements and dosage forms comprising a combination of at least five of the following ingredients: (a) coenzyme Q10; (b) chromium or any pharmaceutically acceptable salt thereof; (c) zinc or any pharmaceutically acceptable salt thereof; (d) magnesium or any pharmaceutically acceptable salt thereof; (e) bitter melon extract; (f) fenugreek extract; (g) cinnamon extract; (h) thiamine; (i) ginger extract; (j) ginseng extract; and (k) copper or any pharmaceutically acceptable salt thereof. The present application also relates to the use of such compositions, nutritional supplements and dosage forms for normalizing glucose levels and for the management of diseases or conditions associated with carbohydrate and lipid metabolism such as diabetes.

### CROSS REFERENCE TO RELATED APPLICATIONS

The application claims the benefit of U.S. Provisional Application No. 63/501,521 filed May 11, 2023 and U.S. Provisional Application No. 63/617,955 filed Jan. 5, 2024. The contents of the referenced provisional applications are incorporated into the present application by reference.

### TECHNICAL FIELD

The present disclosure generally relates to the field of metabolic health, and more particularly to the management of diseases or conditions associated with carbohydrate and fat metabolism such as diabetes.

### BACKGROUND ART

Metabolic Syndrome, or metabolic disease, is a cluster of associated medical conditions and associated pathologies. Typically, the syndrome is associated with at least three of five of the following medical conditions: abdominal obesity, elevated blood pressure, elevated fasting plasma glucose, high serum triglycerides, and low levels of high-density lipoprotein (HDL) levels. An individual with Metabolic Syndrome is at higher risk of developing cardiovascular disease and diabetes, which are leading causes of morbidity and increased risk of mortality.

Type 2 diabetes is a complex disease resulting from both impaired insulin secretion from the pancreas and a failure in the peripheral tissues to respond to normal insulin levels. This inability to adequately manage blood glucose levels is associated with the progressive damage, and ultimate failure, of vital organs, including the eyes, kidneys, nerves, and cardiovascular system. Currently, 463 million individuals worldwide have been diagnosed with Type 2 diabetes, and this population is expected to reach 700 million by 2045. The disease already accounts for as much as 10% of the overall healthcare budget in many countries, and in the United States alone, the total estimated 2017 cost of diagnosed diabetes was $327 billion. This included $237 billion in direct medical costs and $90 billion in reduced productivity.

There is thus a need for therapies to improve carbohydrate and lipid metabolism, and to prevent or treat cardiovascular and metabolic diseases such as diabetes.

The present description refers to a number of documents, the content of which is herein incorporated by reference in their entirety.

### SUMMARY

In an aspect, the present disclosure relates to a composition comprising at least five of the following ingredients:
(a) coenzyme Q10;
(b) chromium or any pharmaceutically acceptable salt thereof;
(c) zinc or any pharmaceutically acceptable salt thereof;
(d) magnesium or any pharmaceutically acceptable salt thereof;
(e) bitter melon extract;
(f) fenugreek extract;
(g) cinnamon extract;
(h) thiamine;
(i) ginger extract;
(j) ginseng extract; and
(k) copper or any pharmaceutically acceptable salt thereof.

In an aspect, the present disclosure relates to a dosage form or nutritional supplement comprising a composition comprising at least five of the following ingredients:
(a) coenzyme Q10;
(b) chromium or any pharmaceutically acceptable salt thereof;
(c) zinc or any pharmaceutically acceptable salt thereof;
(d) magnesium or any pharmaceutically acceptable salt thereof;
(e) bitter melon extract;
(f) fenugreek extract;
(g) cinnamon extract;
(h) thiamine;
(i) ginger extract;
(j) ginseng extract; and
(k) copper or any pharmaceutically acceptable salt thereof.

In an aspect, the present disclosure relates to a composition or dosage form or nutritional supplement comprising the composition, wherein the composition comprises at least five of the following ingredients:
(a) coenzyme Q10;
(b) chromium or any pharmaceutically acceptable salt thereof;
(c) zinc or any pharmaceutically acceptable salt thereof;
(d) magnesium or any pharmaceutically acceptable salt thereof;
(e) bitter melon extract;
(f) fenugreek extract;
(g) cinnamon extract;
(h) thiamine;
(i) ginger extract;
(j) ginseng extract; and
(k) copper or any pharmaceutically acceptable salt thereof, for use in normalizing glucose levels or improving glycemic control in a subject.

In an aspect, the present disclosure relates to the use of a composition or dosage form or nutritional supplement comprising the composition, wherein the composition comprises at least five of the following ingredients:
(a) coenzyme Q10;
(b) chromium or any pharmaceutically acceptable salt thereof;
(c) zinc or any pharmaceutically acceptable salt thereof;
(d) magnesium or any pharmaceutically acceptable salt thereof;
(e) bitter melon extract;
(f) fenugreek extract;
(g) cinnamon extract;
(h) thiamine;
(i) ginger extract;
(j) ginseng extract; and
(k) copper or any pharmaceutically acceptable salt thereof, for the preparation of a medicament for normalizing glucose levels or improving glycemic control in a subject.

In an aspect, the present disclosure relates to a method for normalizing glucose levels or improving glycemic control in a subject comprising administering to the subject an effective amount of a composition or a dosage form or nutritional supplement comprising the composition, wherein the composition comprises at least five of the following ingredients:
(a) coenzyme Q10;
(b) chromium or any pharmaceutically acceptable salt thereof;
(c) zinc or any pharmaceutically acceptable salt thereof;
(d) magnesium or any pharmaceutically acceptable salt thereof;
(e) bitter melon extract;
(f) fenugreek extract;
(g) cinnamon extract;
(h) thiamine;
(i) ginger extract;
(j) ginseng extract; and
(k) copper or any pharmaceutically acceptable salt thereof.

Also disclosed in the context of the present disclosure are embodiments 1 to 45. Embodiment 1 is a composition comprising at least five of the following ingredients: (a) coenzyme Q10; (b) chromium or any pharmaceutically acceptable salt thereof; (c) zinc or any pharmaceutically acceptable salt thereof; (d) magnesium or any pharmaceutically acceptable salt thereof; (e) bitter melon extract; (f) fenugreek extract; (g) cinnamon extract; (h) thiamine; (i) ginger extract; (j) ginseng extract; and (k) copper or any pharmaceutically acceptable salt thereof. Embodiment 2 is the composition of embodiment 1, which comprises at least 6 of ingredients (a)-(k). Embodiment 3 is the composition of embodiment 1, which comprises at least 7 of ingredients (a)-(k). Embodiment 4 is the composition of embodiment 1, which comprises at least 8 of ingredients (a)-(k). Embodiment 5 is the composition of embodiment 1, which comprises at least 9 of ingredients (a)-(k). Embodiment 6 is the composition of embodiment 1, which comprises at least 10 of ingredients (a)-(k). Embodiment 7 is the composition of embodiment 1, which comprises ingredients (a)-(k). Embodiment 8 is the composition of any one of embodiments 1 to 7, wherein coenzyme Q10 is present in an amount of about 5% to about 25% (w/w). Embodiment 9 is the composition of any one of embodiments 1 to 8, wherein chromium is present in an amount of about 0.01% to about 5.0% (w/w). Embodiment 10 is the composition of any one of embodiments 1 to 9, wherein zinc is present in an amount of about 5% to about 15% (w/w). Embodiment 11 is the composition of any one of embodiments 1 to 10, wherein magnesium is present in an amount of about 4% to about 15% (w/w). Embodiment 12 is the composition of any one of embodiments 1 to 11, wherein the bitter melon extract is present in an amount of about 1% to about 15% (w/w). Embodiment 13 is the composition of any one of embodiments 1 to 12, wherein the fenugreek extract is present in an amount of about 1% to about 15% (w/w). Embodiment 14 is the composition of any one of embodiments 1 to 13, wherein the cinnamon extract is present in an amount of about 20% to about 70% (w/w). Embodiment 15 is the composition of any one of embodiments 1 to 14, wherein thiamine is present in an amount of about 10% to about 25% (w/w). Embodiment 16 is the composition of any one of embodiments 1 to 15, wherein the ginger extract is present in an amount of about 10% to about 30% (w/w). Embodiment 17 is the composition of any one of embodiments 1 to 16, wherein the ginseng extract is present in an amount of about 1% to about 20% (w/w). Embodiment 18 is the composition of any one of embodiments 1 to 17, wherein copper is present in an amount of about 0.1% to about 5.0% (w/w). Embodiment 19 is the composition of any one of embodiments 1 to 18, wherein the composition comprises about 30 mg to about 120 mg of coenzyme Q10. Embodiment 20 is the composition of any one of embodiments 1 to 19, wherein the composition comprises about 0.1 mg to about 0.8 mg of chromium. Embodiment 21 is the composition of any one of embodiments 1 to 20, wherein the composition comprises about 20 mg to about 100 mg of zinc. Embodiment 22 is the composition of any one of embodiments 1 to 21, wherein the composition comprises about 10 mg to about 500 mg of magnesium. Embodiment 23 is the composition of any one of embodiments 1 to 22, wherein the 10 composition comprises about 5 mg to about 50 mg of the bitter melon extract. Embodiment 24 is the composition of any one of embodiments 1 to 23, wherein the composition comprises about 5 mg to about 50 mg of the fenugreek extract. Embodiment 25 is the composition of any one of embodiments 1 to 24, wherein the composition comprises about 50 mg to about 500 mg of the cinnamon extract. Embodiment 26 is the composition of any one of embodiments 1 to 25, wherein the composition 15 comprises about 30 mg to about 150 mg of thiamine. Embodiment 27 is the composition of any one of embodiments 1 to 26, wherein the composition comprises about 25 mg to about 125 mg of the ginger. Embodiment 28 is the composition of any one of embodiments 1 to 27, wherein the composition comprises about 5 mg to about 250 mg of the ginseng extract. Embodiment 29 is the composition of any one of embodiments 1 to 28, wherein the composition comprises about 0.5 mg to about 5.0 mg of copper. Embodiment 30 is the composition of any one of embodiments 1 to 29, 20 wherein the composition further comprises one or more pharmaceutically or nutritionally acceptable excipients.

Embodiment 31 is a dosage form or nutritional supplement comprising the composition of any one of embodiments 1 to 30. Embodiment 32 is the dosage form or nutritional supplement of embodiment 31, which is a capsule.

Embodiment 33 is the composition of any one of embodiments 1 to 30 or the dosage form or nutritional supplement of embodiment 31 or 32, for use in at least one of normalizing glucose levels, normalizing dysglycemia, improving fasting glucose, improving HbA1c, improving blood sugar, improving blood glucose, or improving glycemic control in a subject. Embodiment 34 is the composition, dosage form or nutritional supplement for the use according to embodiment 33, wherein the subject suffers from a metabolic condition or metabolic disease. Embodiment 35 is the composition, dosage form or nutritional supplement for the use according to embodiment 34, wherein the metabolic disease is a metabolic syndrome, prediabetes, or Type II diabetes. Embodiment 36 is the composition, dosage form or nutritional supplement for the use according to embodiment 34, wherein the metabolic condition is a metabolic syndrome, prediabetes, or Type II diabetes. Embodiment 37 is the composition, dosage form or nutritional supplement for the use according to any one of embodiments 33 to 35, wherein the dosage form or nutritional supplement is for twice-a-day administration.

Embodiment 38 is the use of the composition of any one of embodiments 1 to 30 or the dosage form or nutritional supplement of embodiment 31 or 32, for the preparation of a medicament for normalizing glucose levels, normalizing dysglycemia, improving fasting glucose, improving HbA1c, improving blood sugar, improving blood glucose or improving glycemic control in a subject. Embodiment 39 is the use according to embodiment 38, wherein the subject suffers from a metabolic condition or a metabolic disease. Embodiment 40 is the use according to embodiment 39, wherein the metabolic condition is metabolic syndrome, prediabetes, or Type II diabetes. Embodiment 41 is the use according to any one of embodiments 38 to 40, wherein the medicament or nutritional supplement is for twice-a-day administration.

Embodiment 42 is a method for normalizing glucose levels, normalizing dysglycemia, improving fasting glucose, improving HbA1c, improving blood sugar, improving blood glucose, or improving glycemic control in a subject comprising administering to the subject an effective amount of the composition of any one of embodiments 1 to 30 or the dosage form or nutritional supplement of embodiment 31 or 32. Embodiment 43 is the method of embodiment 42, wherein the subject suffers from a metabolic condition or a metabolic disease. Embodiment 44 is the method of embodiment 43, wherein the metabolic condition is metabolic syndrome, prediabetes, or Type II diabetes. Embodiment 45 is the method of any one of embodiments 42 to 44, wherein the dosage form is administered twice-a-day.

Other objects, advantages and features of the present disclosure will become more apparent upon reading of the following non-restrictive description of specific embodiments thereof, given by way of example only with reference to the accompanying drawings.

### BRIEF DESCRIPTION OF DRAWINGS

In the appended drawings:

FIG. 1 is a graph depicting the change in hemoglobin A1c levels from baseline to week 12 in the placebo and treatment groups.

FIG. 2 is a graph depicting the average glycosylated hemoglobin (HbA1c) at randomisation and after 3 months of treatment with a formulation according to the present disclosure (Treatment) or Placebo.

FIG. 3 is a graph depicting the 3-day fasting glucose average at randomisation and after 3 months of treatment with a formulation according to the present disclosure (Treatment) or Placebo.

FIG. 4 is a graph depicting the weight at randomisation and after 3 months of treatment with a formulation according to the present disclosure (Treatment) or Placebo.

FIG. 5 is a graph depicting the average total cholesterol at randomisation and after 3 months of treatment with a formulation according to the present disclosure (Treatment) or Placebo.

FIG. 6 is a graph depicting the average triglyceride at randomisation and after 3 months of treatment with a formulation according to the present disclosure (Treatment) or Placebo.

FIG. 7 is a graph depicting the average High-Density Lipoprotein (HDL) at randomisation and after 3 months of treatment with a formulation according to the present disclosure (Treatment) or Placebo.

FIG. 8 is a graph depicting the average Low-Density Lipoprotein (HDL) at randomisation and after 3 months of treatment with a formulation according to the present disclosure (Treatment) or Placebo.

FIG. 9 is a graph depicting the average Aspartate Aminotransferase (AST) at randomisation and after 3 months of treatment with a formulation according to the present disclosure (Treatment) or Placebo.

FIG. 10 is a graph depicting the average Alanine Aminotransferase (ALT) at randomisation and after 3 months of treatment with a formulation according to the present disclosure (Treatment) or Placebo.

FIG. 11 is a graph depicting the average Gamma-Glutamyl Transpeptidase (GGT) at randomisation and after 3 months of treatment with a formulation according to the present disclosure (Treatment) or Placebo.

FIG. 12 is a graph depicting the average creatinine at randomisation and after 3 months of treatment with a formulation according to the present disclosure (Treatment) t or Placebo.

FIG. 13 is a graph depicting the average estimated Glomerular filtration Rate (eGFR) at randomisation and after 3 months of treatment with a formulation according to the present disclosure (Treatment) or Placebo.

FIG. 14 is a graph depicting the average urinary Albumine-to-Creatinine Ratio (uACR) at randomisation and after 3 months of treatment with a formulation according to the present disclosure (Treatment) or Placebo.

### DETAILED DISCLOSURE

The use of the terms “a” and “an” and “the” and similar referents in the context of describing the technology (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

All methods described herein can be performed in any suitable order unless otherwise indicated herein or otherwise clearly contradicted by context.

The use of any and all examples, or exemplary language (“e.g.”, “such as”) provided herein, is intended merely to better illustrate embodiments of the claimed technology and does not pose a limitation on the scope unless otherwise claimed.

No language in the specification should be construed as indicating any non-claimed element as essential to the practice of embodiments of the claimed technology.

Herein, the term “about” has its ordinary meaning. The term “about” is used to indicate that a value includes an inherent variation of error for the device or the method being employed to determine the value, or encompass values close to the recited values, for example within 10% of the recited values (or range of values).

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

Where features or aspects of the disclosure are described in terms of Markush groups or list of alternatives, those skilled in the art will recognize that the disclosure is also thereby described in terms of any individual member, or subgroup of members, of the Markush group or list of alternatives.

Unless specifically defined otherwise, all technical and scientific terms used herein shall be taken to have the same meaning as commonly understood by one of ordinary skill in the art (e.g., in pharmacy and pharmacology).

The present inventors have developed compositions comprising a mixture of ingredients that have complementary/synergistic effects on the regulation of glucose metabolism. A representative composition was shown to significantly lower hemoglobin A1c, a marker of blood glucose level, and to have a positive effect on body weight, following administration to type 2 diabetic subjects over a period of 12 weeks.

The present disclosure provides a composition comprising at least five of the following ingredients:
(a) coenzyme Q10 ([(2E,6E,10E,14E,18E,22E,26E,30E,34E)-3,7,11,15,19,23,27,31,35,39-Decamethyltetraconta-2,6, 10,14, 18,22,26,30,34,38-decaenyl]-5,6-dimethoxy-3-methylcyclohexa-2,5-diene-1,4-dione);
(b) chromium (e.g., chromium picolinate);
(c) zinc (e.g., zinc oxide);
(d) magnesium (e.g., magnesium sulfate);
(e) bitter melon extract (Momordica charantia);
(f) fenugreek extract (Trigonella foenum-graecum);
(g) cinnamon extract (Cinnamomum aromaticum);
(h) thiamine (vitamin B1);
(i) ginger extract (Zingiber officinale);
(j) ginseng extract (Panax quinquefolius); and
(k) copper (e.g., copper citrate).

In an embodiment, the composition comprises ingredient (a). In an embodiment, the composition comprises ingredient (b). In an embodiment, the composition comprises ingredient (c). In an embodiment, the composition comprises ingredient (d). In an embodiment, the composition comprises ingredient (e). In an embodiment, the composition comprises ingredient (f). In an embodiment, the composition comprises ingredient (g). In an embodiment, the composition comprises ingredient (h). In an embodiment, the composition comprises ingredient (i). In an embodiment, the composition comprises ingredient (j). In an embodiment, the composition comprises ingredient (k). In an embodiment, the composition comprises ingredients (b), (e), (f), (g) and (i). In an embodiment, the composition comprises at least 6 of ingredients (a)-(k). In an embodiment, the composition comprises at least 7 of ingredients (a)-(k). In an embodiment, the composition comprises at least 8 of ingredients (a)-(k). In an embodiment, the composition comprises at least 9 of ingredients (a)-(k). In an embodiment, the composition comprises at least 10 of ingredients (a)-(k). In an embodiment, the composition comprises ingredients (a)-(k).

The above metals (magnesium, zinc, chromium and copper) are available in many forms, including physiologically/pharmaceutically acceptable salts. Suitable physiologically acceptable salts of the above metals with organic acids include salts with orotic acid, aspartic acid, gluconic acid, picolinic acid, tartaric acid, citric acid, lactic acid, acetic acid, fumaric acid, maleic acid, malic acid, ascorbic acid, succinic acid, and amino acids, for example glycine, glutamine or cysteine.

Magnesium is available in many forms, including pharmaceutically acceptable of salts such as magnesium adipate, magnesium aspartate, magnesium bisglycinate, magnesium citrate, magnesium gluconate, magnesium glutamate, magnesium glycinate, magnesium lactate, magnesium L-threonate, magnesium lysinate, magnesium malate, magnesium orotate, magnesium picolinate, magnesium sulfate, magnesium succinate or magnesium taurate. In an embodiment, the composition comprises magnesium in the form of magnesium sulfate.

Zinc is available in many forms, including pharmaceutically acceptable zinc salts such as zinc acetate, zinc aspartate, zinc bisglycinate, zinc chloride, zinc citrate, zinc gluconate, zinc glycinate, magnesium lactate, zinc lysinate, zinc malate, zinc monomethionine, zinc orotate, zinc oxide, zinc picolinate or zinc sulfate. In an embodiment, the composition comprises zinc in the form of zinc oxide.

Many forms of copper are known to those skilled in the art, including pharmaceutically acceptable copper salts such as copper aspartate, copper bisglycinate, copper chloride, copper citrate, copper gluconate, copper glycinate, copper lysinate, copper orotate or copper picolinate. In an embodiment, the composition comprises copper in the form of copper citrate.

Chromium is available in many forms, including pharmaceutically acceptable chromium salts such as chromium chloride, chromium citrate, chromium picolinate, chromium nicotinate and chromium polynicotinate. In an embodiment, the composition comprises chromium in the form of chromium picolinate.

In an embodiment, coenzyme Q10 is present in an amount of about 5% to about 25% (w/w). In further embodiments, coenzyme Q10 is present in an amount of about 5% to about 24%, about 5% to about 23%, about 5% to about 22%, about 5% to about 21%, about 5% to about 20%, about 5% to about 19%, about 5% to about 18%, about 5% to about 17%, about 5% to about 16%, about 5% to about 15%, about 6% to about 25%, about 6% to about 24%, about 6% to about 23%, about 6% to about 22%, about 6% to about 21%, about 6% to about 20%, about 6% to about 19%, about 6% to about 18%, about 6% to about 17%, about 6% to about 16%, about 6% to about 15%, about 7% to about 25%, about 7% to about 24%, about 7% to about 23%, about 7% to about 22%, about 7% to about 21%, about 7% to about 20%, about 7% to about 19%, about 7% to about 18%, about 7% to about 17%, about 7% to about 16%, about 7% to about 15%, about 8% to about 25%, about 8% to about 24%, about 8% to about 23%, about 8% to about 22%, about 8% to about 21%, about 8% to about 20%, about 8% to about 19%, about 8% to about 18%, about 8% to about 17%, about 8% to about 16%, about 8% to about 15%, about 9% to about 25%, about 9% to about 24%, about 9% to about 23%, about 9% to about 22%, about 9% to about 21%, about 9% to about 20%, about 9% to about 19%, about 9% to about 18%, about 9% to about 17%, about 9% to about 16%, about 9% to about 15%, about 10% to about 25%, about 10% to about 24%, about 10% to about 23%, about 10% to about 22%, about 10% to about 21%, about 10% to about 20%, about 10% to about 19%, about 10% to about 18%, about 10% to about 17%, about 10% to about 16%, about 10% to about 15%, about 11% to about 25%, about 11% to about 24%, about 11% to about 23%, about 11% to about 22%, about 11% to about 21%, about 11% to about 20%, about 11% to about 19%, about 11% to about 18%, about 11% to about 17%, about 11% to about 16%, about 11% to about 15%, about 12% to about 25%, about 12% to about 24%, about 12% to about 23%, about 12% to about 22%, about 12% to about 21%, about 12% to about 20%, about 12% to about 19%, about 12% to about 18%, about 12% to about 17%, about 12% to about 16%, about 12% to about 15%, about 13% to about 25%, about 13% to about 24%, about 13% to about 23%, about 13% to about 22%, about 13% to about 21%, about 13% to about 20%, about 13% to about 19%, about 13% to about 18%, about 13% to about 17%, about 13% to about 16%, about 13% to about 15%, about 14% to about 25%, about 14% to about 24%, about 14% to about 23%, about 14% to about 22%, about 14% to about 21%, about 14% to about 20%, about 14% to about 19%, about 14% to about 18%, about 14% to about 17%, about 14% to about 16%, or about 14% to about 15% (w/w).

In an embodiment, chromium is present in an amount of about 0.01% to about 5.0% (w/w). In further embodiments, chromium is present in an amount of about 0.01% to about 4.9%, about 0.01% to about 4.8%, about 0.01% to about 4.7%, about 0.01% to about 4.6%, about 0.01% to about 4.5%, about 0.01% to about 4.4%, about 0.01% to about 4.3%, about 0.01% to about 4.2%, about 0.01% to about 4.1%, about 0.01% to about 4.0%, about 0.01% to about 3.9%, about 0.01% to about 3.8%, about 0.01% to about 3.7%, about 0.01% to about 3.6%, about 0.01% to about 3.5%, about 0.01% to about 3.4%, about 0.01% to about 3.3%, about 0.01% to about 3.2%, about 0.01% to about 3.1%, about 0.01% to about 3.0%, about 0.01% to about 2.9%, about 0.01% to about 2.8%, about 0.01% to about 2.7%, about 0.01% to about 2.6%, about 0.01% to about 2.5%, about 0.01% to about 2.4%, about 0.01% to about 2.3%, about 0.01% to about 2.2%, about 0.01% to about 2.1%, about 0.01% to about 2.0%, about 0.01% to about 1.9%, about 0.01% to about 1.8%, about 0.01% to about 1.7%, about 0.01% to about 1.5%, about 0.01% to about 1.4%, about 0.01% to about 1.3%, about 0.01% to about 1.2%, about 0.01% to about 1.1%, about 0.01% to about 1.0%, about 0.02% to about 4.9%, about 0.02% to about 4.8%, about 0.02% to about 4.7%, about 0.02% to about 4.6%, about 0.02% to about 4.5%, about 0.02% to about 4.4%, about 0.02% to about 4.3%, about 0.02% to about 4.2%, about 0.02% to about 4.1%, about 0.02% to about 4.0%, about 0.02% to about 3.9%, about 0.02% to about 3.8%, about 0.02% to about 3.7%, about 0.02% to about 3.6%, about 0.02% to about 3.5%, about 0.02% to about 3.4%, about 0.02% to about 3.3%, about 0.02% to about 3.2%, about 0.02% to about 3.1%, about 0.02% to about 3.0%, about 0.02% to about 2.9%, about 0.02% to about 2.8%, about 0.02% to about 2.7%, about 0.02% to about 2.6%, about 0.02% to about 2.5%, about 0.02% to about 2.4%, about 0.02% to about 2.3%, about 0.02% to about 2.2%, about 0.02% to about 2.1%, about 0.02% to about 2.0%, about 0.02% to about 1.9%, about 0.02% to about 1.8%, about 0.02% to about 1.7%, about 0.02% to about 1.5%, about 0.02% to about 1.4%, about 0.02% to about 1.3%, about 0.02% to about 1.2%, about 0.02% to about 1.1%, about 0.02% to about 1.0%, about 0.05% to about 5.0%, about 0.05% to about 4.9%, about 0.05% to about 4.8%, about 0.05% to about 4.7%, about 0.05% to about 4.6%, about 0.05% to about 4.5%, about 0.05% to about 4.4%, about 0.05% to about 4.3%, about 0.05% to about 4.2%, about 0.05% to about 4.1%, about 0.05% to about 4.0%, about 0.05% to about 3.9%, about 0.05% to about 3.8%, about 0.05% to about 3.7%, about 0.05% to about 3.6%, about 0.05% to about 3.5%, about 0.05% to about 3.4%, about 0.05% to about 3.3%, about 0.05% to about 3.2%, about 0.05% to about 3.1%, about 0.05% to about 3.0%, about 0.05% to about 2.9%, about 0.05% to about 2.8%, about 0.05% to about 2.7%, about 0.05% to about 2.6%, about 0.05% to about 2.5%, about 0.05% to about 2.4%, about 0.05% to about 2.3%, about 0.05% to about 2.2%, about 0.05% to about 2.1%, about 0.05% to about 2.0%, about 0.05% to about 1.9%, about 0.05% to about 1.8%, about 0.05% to about 1.7%, about 0.05% to about 1.5%, about 0.05% to about 1.4%, about 0.05% to about 1.3%, about 0.05% to about 1.2%, about 0.05% to about 1.1%, about 0.05% to about 1.0%, about 1.0% to about 5.0%, about 1.0% to about 4.9%, about 1.0% to about 4.8%, about 1.0% to about 4.7%, about 1.0% to about 4.6%, about 1.0% to about 4.5%, about 1.0% to about 4.4%, about 1.0% to about 4.3%, about 1.0% to about 4.2%, about 1.0% to about 4.1%, about 1.0% to about 4.0%, about 1.0% to about 3.9%, about 1.0% to about 3.8%, about 1.0% to about 3.7%, about 1.0% to about 3.6%, about 1.0% to about 3.5%, about 1.0% to about 3.4%, about 1.0% to about 3.3%, about 1.0% to about 3.2%, about 1.0% to about 3.1%, about 1.0% to about 3.0%, about 1.0% to about 2.9%, about 1.0% to about 2.8%, about 1.0% to about 2.7%, about 1.0% to about 2.6%, about 1.0% to about 2.5%, about 1.0% to about 2.4%, about 1.0% to about 2.3%, about 1.0% to about 2.2%, about 1.0% to about 2.1%, about 1.0% to about 2.0%, about 1.0% to about 1.9%, about 1.0% to about 1.8%, about 1.0% to about 1.7%, about 1.0% to about 1.5%, about 0.03% to about 0.09%, about 0.05% to about 0.08%, about 0.06% to about 0.08% or about 0.07% to about 0.075% (w/w). In an embodiment, the chromium advantageously comprises chromium picolinate.

In an embodiment, zinc is present in an amount of about 5% to about 15% (w/w). In further embodiments, zinc is present in an amount of about 5% to about 14%, about 5% to about 13%, about 5% to about 12%, about 5% to about 11%, about 5% to about 10%, about 5% to about 9%, about 5% to about 8%, about 5% to about 7%, about 5% to about 6%, about 6% to about 15%, about 6% to about 14%, about 6% to about 13%, about 6% to about 12%, about 6% to about 11%, about 6% to about 10%, about 6% to about 9%, about 6% to about 8%, about 6% to about 7%, about 7% to about 15%, about 7% to about 14%, about 7% to about 13%, about 7% to about 12%, about 7% to about 11%, about 7% to about 10%, about 7% to about 9%, about 7% to about 8%, about 8% to about 15%, about 8% to about 14%, about 8% to about 13%, about 8% to about 12%, about 8% to about 11%, about 8% to about 10%, about 8% to about 9%, about 9% to about 15%, about 9% to about 14%, about 9% to about 13%, about 9% to about 12%, about 9% to about 11%, about 9% to about 10%, about 10% to about 15%, about 10% to about 14%, about 10% to about 13%, about 10% to about 12%, about 10% to about 11%, about 11% to about 15%, about 11% to about 14%, about 11% to about 13%, about 11% to about 12%, about 12% to about 15%, about 12% to about 14%, about 12% to about 13%, about 13% to about 15% or about 13% to about 14% (w/w).

In an embodiment, magnesium is present in an amount of about 4% to about 15% (w/w). In further embodiments magnesium is present in an amount of about 5% to about 14%, about 5% to about 13%, about 5% to about 12%, about 5% to about 11%, about 5% to about 10%, about 5% to about 9%, about 5% to about 8%, about 5% to about 7%, about 5% to about 6%, about 6% to about 15%, about 6% to about 14%, about 6% to about 13%, about 6% to about 12%, about 6% to about 11%, about 6% to about 10%, about 6% to about 9%, about 6% to about 8%, about 6% to about 7%, about 7% to about 15%, about 7% to about 14%, about 7% to about 13%, about 7% to about 12%, about 7% to about 11%, about 7% to about 10%, about 7% to about 9%, about 7% to about 8%, about 8% to about 15%, about 8% to about 14%, about 8% to about 13%, about 8% to about 12%, about 8% to about 11%, about 8% to about 10%, about 8% to about 9%, about 9% to about 15%, about 9% to about 14%, about 9% to about 13%, about 9% to about 12%, about 9% to about 11%, about 9% to about 10%, about 10% to about 15%, about 10% to about 14%, about 10% to about 13%, about 10% to about 12%, about 10% to about 11%, about 11% to about 15%, about 11% to about 14%, about 11% to about 13%, about 11% to about 12%, about 12% to about 15%, about 12% to about 14%, about 12% to about 13%, about 13% to about 15%, about 13% to about 14%, about 4% to about 14%, about 4% to about 13%, about 4% to about 12%, about 4% to about 11%, about 4% to about 10%, about 4% to about 9%, about 4% to about 8%, about 4% to about 7% or about 4% to about 6% (w/w).

In an embodiment, the bitter melon extract is present in an amount of about 1% to about 15% (w/w). In further embodiments bitter melon extract is present in an amount of about 5% to about 14%, about 5% to about 13%, about 5% to about 12%, about 5% to about 11%, about 5% to about 10%, about 5% to about 9%, about 5% to about 8%, about 5% to about 7%, about 5% to about 6%, about 6% to about 15%, about 6% to about 14%, about 6% to about 13%, about 6% to about 12%, about 6% to about 11%, about 6% to about 10%, about 6% to about 9%, about 6% to about 8%, about 6% to about 7%, about 7% to about 15%, about 7% to about 14%, about 7% to about 13%, about 7% to about 12%, about 7% to about 11%, about 7% to about 10%, about 7% to about 9%, about 7% to about 8%, about 8% to about 15%, about 8% to about 14%, about 8% to about 13%, about 8% to about 12%, about 8% to about 11%, about 8% to about 10%, about 8% to about 9%, about 9% to about 15%, about 9% to about 14%, about 9% to about 13%, about 9% to about 12%, about 9% to about 11%, about 9% to about 10%, about 10% to about 15%, about 10% to about 14%, about 10% to about 13%, about 10% to about 12%, about 10% to about 11%, about 11% to about 15%, about 11% to about 14%, about 11% to about 13%, about 11% to about 12%, about 12% to about 15%, about 12% to about 14%, about 12% to about 13%, about 13% to about 15%, about 13% to about 14%, about 4% to about 14%, about 4% to about 13%, about 4% to about 12%, about 4% to about 11%, about 4% to about 10%, about 4% to about 9%, about 4% to about 8%, about 4% to about 7%, about 4% to about 6%, about 1% to about 14%, about 1% to about 13%, about 1% to about 12%, about 1% to about 11%, about 1% to about 10%, about 1% to about 9%, about 1% to about 8%, about 1% to about 7%, about 1% to about 6%, about 1% to about 5%, about 2% to about 15%, about 2% to about 14%, about 2% to about 13%, about 2% to about 12%, about 2% to about 11%, about 2% to about 10%, about 2% to about 9%, about 2% to about 8%, about 2% to about 7%, about 2% to about 6%, about 2% to about 5%, about 3% to about 15%, about 3% to about 14%, about 3% to about 13%, about 3% to about 12%, about 3% to about 11%, about 3% to about 10%, about 3% to about 9%, about 3% to about 8%, about 3% to about 7%, about 3% to about 6% or about 3% to about 5% (w/w).

In an embodiment, the fenugreek extract is present in an amount of about 1% to about 15% (w/w). In further embodiments fenugreek extract is present in an amount of about 5% to about 14%, about 5% to about 13%, about 5% to about 12%, about 5% to about 11%, about 5% to about 10%, about 5% to about 9%, about 5% to about 8%, about 5% to about 7%, about 5% to about 6%, about 6% to about 15%, about 6% to about 14%, about 6% to about 13%, about 6% to about 12%, about 6% to about 11%, about 6% to about 10%, about 6% to about 9%, about 6% to about 8%, about 6% to about 7%, about 7% to about 15%, about 7% to about 14%, about 7% to about 13%, about 7% to about 12%, about 7% to about 11%, about 7% to about 10%, about 7% to about 9%, about 7% to about 8%, about 8% to about 15%, about 8% to about 14%, about 8% to about 13%, about 8% to about 12%, about 8% to about 11%, about 8% to about 10%, about 8% to about 9%, about 9% to about 15%, about 9% to about 14%, about 9% to about 13%, about 9% to about 12%, about 9% to about 11%, about 9% to about 10%, about 10% to about 15%, about 10% to about 14%, about 10% to about 13%, about 10% to about 12%, about 10% to about 11%, about 11% to about 15%, about 11% to about 14%, about 11% to about 13%, about 11% to about 12%, about 12% to about 15%, about 12% to about 14%, about 12% to about 13%, about 13% to about 15%, about 13% to about 14%, about 4% to about 14%, about 4% to about 13%, about 4% to about 12%, about 4% to about 11%, about 4% to about 10%, about 4% to about 9%, about 4% to about 8%, about 4% to about 7%, about 4% to about 6%, about 1% to about 14%, about 1% to about 13%, about 1% to about 12%, about 1% to about 11%, about 1% to about 10%, about 1% to about 9%, about 1% to about 8%, about 1% to about 7%, about 1% to about 6%, about 1% to about 5%, about 2% to about 15%, about 2% to about 14%, about 2% to about 13%, about 2% to about 12%, about 2% to about 11%, about 2% to about 10%, about 2% to about 9%, about 2% to about 8%, about 2% to about 7%, about 2% to about 6%, about 2% to about 5%, about 3% to about 15%, about 3% to about 14%, about 3% to about 13%, about 3% to about 12%, about 3% to about 11%, about 3% to about 10%, about 3% to about 9%, about 3% to about 8%, about 3% to about 7%, about 3% to about 6% or about 3% to about 5% (w/w).

In an embodiment, the cinnamon extract is present in an amount of about 20% to about 70% (w/w). In further embodiments cinnamon extract is present in an amount of about 21% to about 70%, about 22% to about 70%, about 23% to about 70%, about 24% to about 70%, about 25% to about 70%, about 25% to about 70%, about 26% to about 70%, about 27% to about 70%, about 28% to about 70%, about 29% to about 70%, about 30% to about 70%, about 31% to about 70%, about 32% to about 70%, about 33% to about 70%, about 34% to about 70%, about 35% to about 70%, about 36% to about 70%, about 37% to about 70%, about 38% to about 70%, about 39% to about 70% about 40% to about 70%, about 41% to about 70%, about 42% to about 70%, about 43% to about 70%, about 44% to about 70%, about 45% to about 70%, about 46% to about 70%, about 47% to about 70%, about 48% to about 70%, about 49% to about 70%, about 50% to about 70%, about 51% to about 70%, about 52% to about 70%, about 53% to about 70%, about 54% to about 70%, about 55% to about 70%, about 56% to about 70%, about 57% to about 70%, about 58% to about 70%, about 59% to about 70%, about 60% to about 70%, about 61% to about 70%, about 62% to about 70%, about 63% to about 70%, about 64% to about 70%, about 65% to about 70%, about 66% to about 70%, about 67% to about 70%, about 68% to about 70%, about 69% to about 70%, 20% to about 65%, 21% to about 65%, about 22% to about 65%, about 23% to about 65%, about 24% to about 65%, about 25% to about 65%, about 25% to about 65%, about 26% to about 65%, about 27% to about 65%, about 28% to about 65%, about 29% to about 65%, about 30% to about 65%, about 31% to about 65%, about 32% to about 65%, about 33% to about 65%, about 34% to about 65%, about 35% to about 65%, about 36% to about 65%, about 37% to about 65%, about 38% to about 65%, about 39% to about 65% about 40% to about 65%, about 41% to about 65%, about 42% to about 65%, about 43% to about 65%, about 44% to about 65%, about 45% to about 65%, about 46% to about 65%, about 47% to about 65%, about 48% to about 65%, about 49% to about 65%, about 50% to about 65%, about 51% to about 65%, about 52% to about 65%, about 53% to about 65%, about 54% to about 65%, about 55% to about 65%, about 56% to about 65%, about 57% to about 65%, about 58% to about 65%, about 59% to about 65%, about 60% to about 65%, about 61% to about 65%, about 62% to about 65%, about 63% to about 65%, about 64% to about 65%, 20% to about 60%, 21% to about 60%, about 22% to about 60%, about 23% to about 60%, about 24% to about 60%, about 25% to about 60%, about 25% to about 60%, about 26% to about 60%, about 27% to about 60%, about 28% to about 60%, about 29% to about 60%, about 30% to about 60%, about 31% to about 60%, about 32% to about 60%, about 33% to about 60%, about 34% to about 60%, about 35% to about 60%, about 36% to about 60%, about 37% to about 60%, about 38% to about 60%, about 39% to about 60% about 40% to about 60%, about 41% to about 60%, about 42% to about 60%, about 43% to about 60%, about 44% to about 60%, about 45% to about 60%, about 46% to about 60%, about 47% to about 60%, about 48% to about 60%, about 49% to about 60%, about 50% to about 60%, about 51% to about 60%, about 52% to about 60%, about 53% to about 60%, about 54% to about 60%, about 55% to about 60%, about 56% to about 60%, about 57% to about 60%, about 58% to about 60%, about 59% to about 60%, 20% to about 55%, 21% to about 55%, about 22% to about 55%, about 23% to about 55%, about 24% to about 55%, about 25% to about 55%, about 25% to about 55%, about 26% to about 55%, about 27% to about 55%, about 28% to about 55%, about 29% to about 55%, about 30% to about 55%, about 31% to about 55%, about 32% to about 55%, about 33% to about 55%, about 34% to about 55%, about 35% to about 55%, about 36% to about 55%, about 37% to about 55%, about 38% to about 55%, about 39% to about 55% about 40% to about 55%, about 41% to about 55%, about 42% to about 55%, about 43% to about 55%, about 44% to about 55%, about 45% to about 55%, about 46% to about 55%, about 47% to about 55%, about 48% to about 55%, about 49% to about 55%, about 50% to about 55%, 20% to about 50%, 21% to about 50%, about 22% to about 50%, about 23% to about 50%, about 24% to about 50%, about 25% to about 50%, about 25% to about 50%, about 26% to about 50%, about 27% to about 50%, about 28% to about 50%, about 29% to about 50%, about 30% to about 50%, about 31% to about 50%, about 32% to about 50%, about 33% to about 50%, about 34% to about 50%, about 35% to about 50%, about 36% to about 50%, about 37% to about 50%, about 38% to about 50%, about 39% to about 50% about 40% to about 50%, about 41% to about 50%, about 42% to about 50%, about 43% to about 50%, about 44% to about 50%, about 45% to about 50%, about 46% to about 50%, about 47% to about 50%, about 48% to about 50%, about 49% to about 50%, about 50% to about 52%, 20% to about 45%, 21% to about 45%, about 22% to about 45%, about 23% to about 45%, about 24% to about 45%, about 25% to about 45%, about 25% to about 45%, about 26% to about 45%, about 27% to about 45%, about 28% to about 45%, about 29% to about 45%, about 30% to about 45%, about 31% to about 45%, about 32% to about 45%, about 33% to about 45%, about 34% to about 45%, about 35% to about 45%, about 36% to about 45%, about 37% to about 45%, about 38% to about 45%, about 39% to about 45% about 40% to about 45%, about 41% to about 45%, about 42% to about 45%, about 43% to about 45%, about 44% to about 45%, 20% to about 40%, 21% to about 40%, about 22% to about 40%, about 23% to about 40%, about 24% to about 40%, about 25% to about 40%, about 25% to about 40%, about 26% to about 40%, about 27% to about 40%, about 28% to about 40%, about 29% to about 40%, about 30% to about 40%, about 31% to about 40%, about 32% to about 40%, about 33% to about 40%, about 34% to about 40%, about 35% to about 40%, about 36% to about 40%, about 37% to about 40%, about 38% to about 40%, about 39% to about 40%, 20% to about 35%, 21% to about 35%, about 22% to about 35%, about 23% to about 35%, about 24% to about 35%, about 25% to about 35%, about 25% to about 35%, about 26% to about 35%, about 27% to about 35%, about 28% to about 35%, about 29% to about 35%, about 30% to about 35%, about 31% to about 35%, about 32% to about 35%, about 33% to about 35%, about 34% to about 35%, 20% to about 30%, 21% to about 30%, about 22% to about 30%, about 23% to about 30%, about 24% to about 30%, about 25% to about 30%, about 25% to about 30%, about 26% to about 30%, about 27% to about 30%, about 28% to about 30% or about 29% to about 30% (w/w)/. In an embodiment, the cinnamon extract advantageously comprises a Cassia extract.

In an embodiment, thiamine is present in an amount of about 10% to about 25% (w/w). In further embodiments thiamine is present in an amount of about 10% to about 24%, about 10% to about 23%, about 10% to about 22%, about 10% to about 21%, about 10% to about 20%, about 10% to about 19%, about 10% to about 18%, about 10% to about 17%, about 10% to about 16%, about 10% to about 15%, about 11% to about 25%, about 11% to about 24%, about 11% to about 23%, about 11% to about 22%, about 11% to about 21%, about 11% to about 20%, about 11% to about 19%, about 11% to about 18%, about 11% to about 17%, about 11% to about 16%, about 11% to about 15%, about 12% to about 25%, about 12% to about 24%, about 12% to about 23%, about 12% to about 22%, about 12% to about 21%, about 12% to about 20%, about 12% to about 19%, about 12% to about 18%, about 12% to about 17%, about 12% to about 16%, about 12% to about 15%, about 13% to about 25%, about 13% to about 24%, about 13% to about 23%, about 13% to about 22%, about 13% to about 21%, about 13% to about 20%, about 13% to about 19%, about 13% to about 18%, about 13% to about 17%, about 13% to about 16%, about 13% to about 15%, about 14% to about 25%, about 14% to about 24%, about 14% to about 23%, about 14% to about 22%, about 14% to about 21%, about 14% to about 20%, about 14% to about 19%, about 14% to about 18%, about 14% to about 17%, about 14% to about 16% or about 14% to about 15% (w/w).

In an embodiment, the ginger extract is present in an amount of about 10% to about 30% (w/w). In further embodiments ginger extractis is present in an amount of about 10% to about 29%, about 10% to about 28%, about 10% to about 27%, about 10%, to about 26%, 10% to about 25%, about 10% to about 24%, about 10% to about 23%, about 10% to about 22%, about 10% to about 21%, about 10% to about 20%, about 10% to about 19%, about 10% to about 18%, about 10% to about 17%, about 10% to about 16%, about 10% to about 15%, about 11% to about 30%, about 11% to about 29%, about 11% to about 28%, about 11% to about 27%, about 11%, to about 26%, about 11% to about 25%, about 11% to about 24%, about 11% to about 23%, about 11% to about 22%, about 11% to about 21%, about 11% to about 20%, about 11% to about 19%, about 11% to about 18%, about 11% to about 17%, about 11% to about 16%, about 11% to about 15%, about 12% to about 30%, about 12% to about 29%, about 12% to about 28%, about 12% to about 27%, about 12%, to about 26%, about 12% to about 25%, about 12% to about 24%, about 12% to about 23%, about 12% to about 22%, about 12% to about 21%, about 12% to about 20%, about 12% to about 19%, about 12% to about 18%, about 12% to about 17%, about 12% to about 16%, about 12% to about 15%, about 13% to about 30%, about 13% to about 29%, about 13% to about 28%, about 13% to about 27%, about 13%, to about 26%, about 13% to about 25%, about 13% to about 24%, about 13% to about 23%, about 13% to about 22%, about 13% to about 21%, about 13% to about 20%, about 13% to about 19%, about 13% to about 18%, about 13% to about 17%, about 13% to about 16%, about 13% to about 15%, about 14% to about 30%, about 14% to about 29%, about 14% to about 28%, about 14% to about 27%, about 14%, to about 26%, about 14% to about 25%, about 14% to about 24%, about 14% to about 23%, about 14% to about 22%, about 14% to about 21%, about 14% to about 20%, about 14% to about 19%, about 14% to about 18%, about 14% to about 17%, about 14% to about 16% or about 14% to about 15% (w/w).

In an embodiment, the ginseng extract is present in an amount of about 1% to about 20% (w/w). In further embodiments ginseng extracts is present in an amount of about 1% to about 19%, about 1% to about 18%, 1% to about 17%, 1% to about 16%, 1% to about 15%, about 1% to about 14%, about 1% to about 13%, about 1% to about 12%, about 1% to about 11%, about 1% to about 10%, about 1% to about 9%, about 1% to about 8%, about 1% to about 7%, about 1% to about 6%, about 1% to about 5%, about 2% to about 20%, about 2% to about 19%, about 2% to about 18%, about 2% to about 17%, about 2% to about 16%, about 2% to about 15%, about 2% to about 14%, about 2% to about 13%, about 2% to about 12%, about 2% to about 11%, about 2% to about 10%, about 2% to about 9%, about 2% to about 8%, about 2% to about 7%, about 2% to about 6%, about 2% to about 5%, about 3% to about 20%, about 3% to about 19%, about 3% to about 18%, about 3% to about 17%, about 3% to about 16%, about 3% to about 15%, about 3% to about 14%, about 3% to about 13%, about 3% to about 12%, about 3% to about 11%, about 3% to about 10%, about 3% to about 9%, about 3% to about 8%, about 3% to about 7%, about 3% to about 6% or about 3% to about 5%, about 4% to about 20%, about 4% to about 19%, about 4% to about 18%, about 4% to about 17%, about 4% to about 16%, about 4% to about 15%, about 4% to about 14%, about 4% to about 13%, about 4% to about 12%, about 4% to about 11%, about 4% to about 10%, about 4% to about 9%, about 4% to about 8%, about 4% to about 7%, about 4% to about 6%, about 5% to about 20%, about 5% to about 19%, about 5% to about 18%, about 5% to about 17%, about 5% to about 16%, about 5% to about 15%, about 5% to about 14%, about 5% to about 13%, about 5% to about 12%, about 5% to about 11%, about 5% to about 10%, about 5% to about 9%, about 5% to about 8%, about 5% to about 7%, about 5% to about 6%, about 6% to about 20%, about 6% to about 19%, about 6% to about 18%, about 6% to about 17%, about 6% to about 16%, about 6% to about 15%, about 6% to about 14%, about 6% to about 13%, about 6% to about 12%, about 6% to about 11%, about 6% to about 10%, about 6% to about 9%, about 6% to about 8%, about 6% to about 7%, about 7% to about 20%, about 7% to about 19%, about 7% to about 18%, about 7% to about 17%, about 7% to about 16%, about 7% to about 15%, about 7% to about 14%, about 7% to about 13%, about 7% to about 12%, about 7% to about 11%, about 7% to about 10%, about 7% to about 9%, about 7% to about 8%, about 8% to about 20%, about 8% to about 19%, about 8% to about 18%, about 8% to about 17%, about 8% to about 16%, about 8% to about 15%, about 8% to about 14%, about 8% to about 13%, about 8% to about 12%, about 8% to about 11%, about 8% to about 10%, about 8% to about 9%, about 9% to about 20%, about 9% to about 19%, about 9% to about 18%, about 9% to about 17%, about 9% to about 16%, about 9% to about 15%, about 9% to about 14%, about 9% to about 13%, about 9% to about 12%, about 9% to about 11%, about 9% to about 10%, about 10% to about 20%, about 10% to about 19%, about 10% to about 18%, about 10% to about 17%, about 10% to about 16%, about 10% to about 15%, about 10% to about 14%, about 10% to about 13%, about 10% to about 12%, about 10% to about 11%, about 11% to about 20%, about 11% to about 19%, about 11% to about 18%, about 11% to about 17%, about 11% to about 16%, about 11% to about 15%, about 11% to about 14%, about 11% to about 13%, about 11% to about 12%, about 12% to about 20%, about 12% to about 19%, about 12% to about 18%, about 12% to about 17%, about 12% to about 16%, about 12% to about 15%, about 12% to about 14%, about 12% to about 13%, about 13% to about 15%, about 13% to about 14%, about 13% to about 20%, about 13% to about 19%, about 13% to about 18%, about 13% to about 17%, about 13% to about 16%, about 13% to about 15%, about 13% to about 14%, about 14% to about 20%, about 14% to about 19%, about 14% to about 18%, about 14% to about 17%, about 14% to about 16%, about 14% to about 15%, about 15% to about 20%, about 15% to about 19%, about 15% to about 18%, about 15% to about 17%, about 15% to about 16%, about 16% to about 20%, about 16% to about 19%, about 16% to about 18%, about 16% to about 17%, about 17% to about 20%, about 17% to about 19%, about 17% to about 18%, about 18% to about 20%, about 18% to about 19% or about 19% to about 20% (w/w).

In an embodiment, the ginseng extract is present in an amount of about 1% to about 20% (w/w). In further embodiments ginseng extract is present in an amount of about 5% to about 14%, about 5% to about 13%, about 5% to about 12%, about 5% to about 11%, about 5% to about 10%, about 5% to about 9%, about 5% to about 8%, about 5% to about 7%, about 5% to about 6%, about 6% to about 15%, about 6% to about 14%, about 6% to about 13%, about 6% to about 12%, about 6% to about 11%, about 6% to about 10%, about 6% to about 9%, about 6% to about 8%, about 6% to about 7%, about 7% to about 15%, about 7% to about 14%, about 7% to about 13%, about 7% to about 12%, about 7% to about 11%, about 7% to about 10%, about 7% to about 9%, about 7% to about 8%, about 8% to about 15%, about 8% to about 14%, about 8% to about 13%, about 8% to about 12%, about 8% to about 11%, about 8% to about 10%, about 8% to about 9%, about 9% to about 15%, about 9% to about 14%, about 9% to about 13%, about 9% to about 12%, about 9% to about 11%, about 9% to about 10%, about 10% to about 15%, about 10% to about 14%, about 10% to about 13%, about 10% to about 12%, about 10% to about 11%, about 11% to about 15%, about 11% to about 14%, about 11% to about 13%, about 11% to about 12%, about 12% to about 15%, about 12% to about 14%, about 12% to about 13%, about 13% to about 15%, about 13% to about 14%, about 4% to about 14%, about 4% to about 13%, about 4% to about 12%, about 4% to about 11%, about 4% to about 10%, about 4% to about 9%, about 4% to about 8%, about 4% to about 7%, about 4% to about 6%, about 1% to about 14%, about 1% to about 13%, about 1% to about 12%, about 1% to about 11%, about 1% to about 10%, about 1% to about 9%, about 1% to about 8%, about 1% to about 7%, about 1% to about 6%, about 1% to about 5%, about 2% to about 15%, about 2% to about 14%, about 2% to about 13%, about 2% to about 12%, about 2% to about 11%, about 2% to about 10%, about 2% to about 9%, about 2% to about 8%, about 2% to about 7%, about 2% to about 6%, about 2% to about 5%, about 3% to about 15%, about 3% to about 14%, about 3% to about 13%, about 3% to about 12%, about 3% to about 11%, about 3% to about 10%, about 3% to about 9%, about 3% to about 8%, about 3% to about 7%, about 3% to about 6% or about 3% to about 5%, about 1% to bout 16%, about 1% to about 17%, about 1% to about 18%, 1% to about 19%, about 2% to bout 16%, about 2% to about 17%, about 2% to about 18%, 2% to about 19%, 2% to about 20%, about 3% to bout 16%, about 3% to about 17%, about 3% to about 18%, 3% to about 19%, 3% to about 20%, about 4% to bout 16%, about 4% to about 17%, about 4% to about 18%, 4% to about 19%, 4% to about 20%, about 5% to bout 16%, about 5% to about 17%, about 5% to about 18%, 5% to about 19%, 5% to about 20%, about 6% to bout 16%, about 6% to about 17%, about 6% to about 18%, 6% to about 19%, 6% to about 20%, about 7% to bout 16%, about 7% to about 17%, about 7% to about 18%, 7% to about 19%, 7% to about 20%, about 8% to bout 16%, about 8% to about 17%, about 8% to about 18%, 8% to about 19%, 8% to about 20%, about 9% to bout 16%, about 9% to about 17%, about 9% to about 18%, 9% to about 19%, 9% to about 20%, about 10% to bout 16%, about 10% to about 17%, about 10% to about 18%, 10% to about 19%, 10% to about 20%, about 11% to bout 16%, about 11% to about 17%, about 11% to about 18%, 11% to about 19%, 11% to about 20%, about 12% to bout 16%, about 12% to about 17%, about 12% to about 18%, 12% to about 19%, 12% to about 20%, about 13% to bout 16%, about 13% to about 17%, about 13% to about 18%, 13% to about 19%, 13% to about 20%, about 14% to bout 16%, about 14% to about 17%, about 14% to about 18%, 14% to about 19%, 14% to about 20%, about 15% to bout 16%, about 15% to about 17%, about 15% to about 18%, 15% to about 19%, 15% to about 20%, about 16% to about 17%, about 16% to about 18%, 16% to about 19%, 16% to about 20%, about 17% to about 18%, 17% to about 19%, 17% to about 20%, about 18% to about 19%, 18% to about 20% or about 19% to about 20% (w/w).

In an embodiment, copper is present in an amount of about 0.1% to about 5.0% (w/w). In further embodiments, copper is present in an amount of about 0.01% to about 4.9%, about 0.01% to about 4.8%, about 0.01% to about 4.7%, about 0.01% to about 4.6%, about 0.01% to about 4.5%, about 0.01% to about 4.4%, about 0.01% to about 4.3%, about 0.01% to about 4.2%, about 0.01% to about 4.1%, about 0.01% to about 4.0%, about 0.01% to about 3.9%, about 0.01% to about 3.8%, about 0.01% to about 3.7%, about 0.01% to about 3.6%, about 0.01% to about 3.5%, about 0.01% to about 3.4%, about 0.01% to about 3.3%, about 0.01% to about 3.2%, about 0.01% to about 3.1%, about 0.01% to about 3.0%, about 0.01% to about 2.9%, about 0.01% to about 2.8%, about 0.01% to about 2.7%, about 0.01% to about 2.6%, about 0.01% to about 2.5%, about 0.01% to about 2.4%, about 0.01% to about 2.3%, about 0.01% to about 2.2%, about 0.01% to about 2.1%, about 0.01% to about 2.0%, about 0.01% to about 1.9%, about 0.01% to about 1.8%, about 0.01% to about 1.7%, about 0.01% to about 1.5%, about 0.01% to about 1.4%, about 0.01% to about 1.3%, about 0.01% to about 1.2%, about 0.01% to about 1.1%, about 0.01% to about 1.0%, about 0.02% to about 4.9%, about 0.02% to about 4.8%, about 0.02% to about 4.7%, about 0.02% to about 4.6%, about 0.02% to about 4.5%, about 0.02% to about 4.4%, about 0.02% to about 4.3%, about 0.02% to about 4.2%, about 0.02% to about 4.1%, about 0.02% to about 4.0%, about 0.02% to about 3.9%, about 0.02% to about 3.8%, about 0.02% to about 3.7%, about 0.02% to about 3.6%, about 0.02% to about 3.5%, about 0.02% to about 3.4%, about 0.02% to about 3.3%, about 0.02% to about 3.2%, about 0.02% to about 3.1%, about 0.02% to about 3.0%, about 0.02% to about 2.9%, about 0.02% to about 2.8%, about 0.02% to about 2.7%, about 0.02% to about 2.6%, about 0.02% to about 2.5%, about 0.02% to about 2.4%, about 0.02% to about 2.3%, about 0.02% to about 2.2%, about 0.02% to about 2.1%, about 0.02% to about 2.0%, about 0.02% to about 1.9%, about 0.02% to about 1.8%, about 0.02% to about 1.7%, about 0.02% to about 1.5%, about 0.02% to about 1.4%, about 0.02% to about 1.3%, about 0.02% to about 1.2%, about 0.02% to about 1.1%, about 0.02% to about 1.0%, about 0.05% to about 5.0%, about 0.05% to about 4.9%, about 0.05% to about 4.8%, about 0.05% to about 4.7%, about 0.05% to about 4.6%, about 0.05% to about 4.5%, about 0.05% to about 4.4%, about 0.05% to about 4.3%, about 0.05% to about 4.2%, about 0.05% to about 4.1%, about 0.05% to about 4.0%, about 0.05% to about 3.9%, about 0.05% to about 3.8%, about 0.05% to about 3.7%, about 0.05% to about 3.6%, about 0.05% to about 3.5%, about 0.05% to about 3.4%, about 0.05% to about 3.3%, about 0.05% to about 3.2%, about 0.05% to about 3.1%, about 0.05% to about 3.0%, about 0.05% to about 2.9%, about 0.05% to about 2.8%, about 0.05% to about 2.7%, about 0.05% to about 2.6%, about 0.05% to about 2.5%, about 0.05% to about 2.4%, about 0.05% to about 2.3%, about 0.05% to about 2.2%, about 0.05% to about 2.1%, about 0.05% to about 2.0%, about 0.05% to about 1.9%, about 0.05% to about 1.8%, about 0.05% to about 1.7%, about 0.05% to about 1.5%, about 0.05% to about 1.4%, about 0.05% to about 1.3%, about 0.05% to about 1.2%, about 0.05% to about 1.1%, about 0.05% to about 1.0%, about 1.0% to about 5.0%, about 1.0% to about 4.9%, about 1.0% to about 4.8%, about 1.0% to about 4.7%, about 1.0% to about 4.6%, about 1.0% to about 4.5%, about 1.0% to about 4.4%, about 1.0% to about 4.3%, about 1.0% to about 4.2%, about 1.0% to about 4.1%, about 1.0% to about 4.0%, about 1.0% to about 3.9%, about 1.0% to about 3.8%, about 1.0% to about 3.7%, about 1.0% to about 3.6%, about 1.0% to about 3.5%, about 1.0% to about 3.4%, about 1.0% to about 3.3%, about 1.0% to about 3.2%, about 1.0% to about 3.1%, about 1.0% to about 3.0%, about 1.0% to about 2.9%, about 1.0% to about 2.8%, about 1.0% to about 2.7%, about 1.0% to about 2.6%, about 1.0% to about 2.5%, about 1.0% to about 2.4%, about 1.0% to about 2.3%, about 1.0% to about 2.2%, about 1.0% to about 2.1%, about 1.0% to about 2.0%, about 1.0% to about 1.9%, about 1.0% to about 1.8%, about 1.0% to about 1.7%, about 1.0% to about 1.5%, about 0.03% to about 0.09%, about 0.05% to about 0.08%, about 0.06% to about 0.08% or about 0.07% to about 0.075% (w/w).

In an embodiment, the composition comprises about 30 mg to about 120 mg of coenzyme Q10. In an embodiment, the composition comprises about 30 mg to about 110 mg of coenzyme Q10. In an embodiment, the composition comprises about 30 mg to about 100 mg of coenzyme Q10. In an embodiment, the composition comprises about 30 mg to about 90 mg of coenzyme Q10. In an embodiment, the composition comprises about 30 mg to about 80 mg of coenzyme Q10. In an embodiment, the composition comprises about 30 mg to about 70 mg of coenzyme Q10. In an embodiment, the composition comprises about 30 mg to about 60 mg of coenzyme Q10. In an embodiment, the composition comprises about 30 mg to about 50 mg of coenzyme Q10. In an embodiment, the composition comprises about 30 mg to about 40 mg of coenzyme Q10. In further embodiments, the composition comprises about 40 mg to about 60 mg, about 40 mg to about 70 mg, about 40 mg to about 80 mg, about 40 mg to about 90 mg, about 40 mg to about 100 mg, about 40 mg to about 110 mg, about 50 mg to about 60 mg, about 50 mg to about 70 mg, about 50 mg to about 80 mg, about 50 mg to about 90 mg, about 50 mg to about 100 mg, about 50 mg to about 110 mg, about 60 mg to about 70 mg, about 60 mg to about 80 mg, about 60 mg to about 90 mg, about 60 mg to about 100 mg, about 60 mg to about 110 mg, about 70 mg to about 80 mg, about 70 mg to about 90 mg, about 70 mg to about 100 mg, about 70 mg to about 110 mg, about 80 mg to about 90 mg, about 80 mg to about 100 mg, about 80 mg to about 110 mg, about 90 mg to about 100 mg, about 90 mg to about 110 mg or about 100 mg to about 110 mg of coenzyme Q10.

In an embodiment, the composition comprises about 0.1 mg to about 0.8 mg of chromium. In an embodiment, the composition comprises about 0.1 mg to about 0.7 mg of chromium. In an embodiment, the composition comprises about 0.1 mg to about 0.6 mg of chromium. In an embodiment, the composition comprises about 0.1 mg to about 0.5 mg of chromium. In further embodiments, the composition comprises about 0.1 mg to about 0.4 mg, about 0.1 mg to about 0.3 mg, about 0.1 mg to about 0.2 mg, about 0.2 mg to about 0.8 mg, about 0.2 mg to about 0.7 mg, about 0.2 mg to about 0.6 mg, about 0.2 mg to about 0.5 mg, about 0.2 mg to about 0.4 mg, about 0.2 mg to about 0.3 mg, about 0.3 mg to about 0.8 mg, about 0.3 mg to about 0.7 mg, about 0.3 mg to about 0.6 mg, about 0.3 mg to about 0.5 mg, about 0.3 mg to about 0.4 mg, about 0.4 mg to about 0.8 mg, about 0.4 mg to about 0.7 mg, about 0.4 mg to about 0.6 mg, about 0.4 mg to about 0.5 mg, about 0.5 mg to about 0.8 mg, about 0.5 mg to about 0.7 mg, about 0.5 mg to about 0.6 mg, about 0.6 mg to about 0.8 mg, about 0.6 mg to about 0.7 mg or about 0.7 mg to about 0.8 mg of chromium. In an embodiment, the chromium advanatageously comprises chromium picolonate.

In an embodiment, the composition comprises about 10 mg to about 100 mg of zinc. In an embodiment, the composition comprises about 10 mg to about 90 mg of zinc. In an embodiment, the composition comprises about 10 mg to about 80 mg of zinc. In an embodiment, the composition comprises about 10 mg to about 70 mg of zinc. In an embodiment, the composition comprises about 10 mg to about 60 mg of zinc. In an embodiment, the composition comprises about 10 mg to about 50 mg of zinc. In an embodiment, the composition comprises about 10 mg to about 40 mg of zinc. In an embodiment, the composition comprises about 10 mg to about 30 mg of zinc. In an embodiment, the composition comprises about 10 mg to about 20 mg of zinc. In further embodiments, the composition comprises about 20 mg to about 100 mg, 20 mg to about 90 mg, 20 mg to about 80 mg, 20 mg to about 70 mg, 20 mg to about 60 mg, 20 mg to about 50 mg, 20 mg to about 40 mg, 20 mg to about 30 mg, 30 mg to about 100 mg, 30 mg to about 90 mg, 30 mg to about 80 mg, 30 mg to about 70 mg, 30 mg to about 60 mg, 30 mg to about 50 mg, 30 mg to about 40 mg, 40 mg to about 100 mg, 40 mg to about 90 mg, 40 mg to about 80 mg, 40 mg to about 70 mg, 40 mg to about 60 mg, 40 mg to about 50 mg, 50 mg to about 100 mg, 50 mg to about 90 mg, 50 mg to about 80 mg, 50 mg to about 70 mg, 50 mg to about 60 mg, 60 mg to about 100 mg, 60 mg to about 90 mg, 60 mg to about 80 mg, 60 mg to about 70 mg, 70 mg to about 100 mg, 70 mg to about 90 mg, 70 mg to about 80 mg, 80 mg to about 100 mg, 80 mg to about 90 mg, 90 mg to about 100 mg, about 35 mg to about 45 mg, about 15 mg, about 25 mg, about 55 mg or about 65 mg of zinc

In an embodiment, the composition comprises about 10 mg to about 500 mg of magnesium. In an embodiment, the composition comprises about 10 mg to about 400 mg of magnesium. In an embodiment, the composition comprises about 10 mg to about 375 mg of magnesium. In an embodiment, the composition comprises about 10 mg to about 350 mg of magnesium. In an embodiment, the composition comprises about 10 mg to about 325 mg of magnesium. In an embodiment, the composition comprises about 10 mg to about 300 mg of magnesium. In an further embodiment, the composition comprises about 10 mg to about 275 mg, about 10 mg to about 250 mg, about 10 mg to about 225 mg, about 10 mg to about 200 mg, about 10 mg to about 175 mg, about 10 mg to about 150 mg, about 10 mg to about 125 mg, about 10 mg to about 100 mg, about 10 mg to about 90 mg, about 10 mg to about 80 mg, about 10 mg to about 70 mg, about 10 mg to about 60 mg, about 10 mg to about 50 mg, about 10 mg to about 40 mg, about 10 mg to about 30 mg, about 10 mg to about 20 mg, about 25 mg to about 500 mg, about 25 mg to about 475 mg, about 25 mg to about 450 mg, about 25 mg to about 425 mg, about 25 mg to about 400 mg, about 25 mg to about 375 mg, about 25 mg to about 350 mg, about 25 mg to about 325 mg, about 25 mg to about 300 mg, about 25 mg to about 275 mg, about 25 mg to about 250 mg, about 25 mg to about 225 mg, about 25 mg to about 200 mg, about 25 mg to about 175 mg, about 25 mg to about 150 mg, about 25 mg to about 125 mg, about 25 mg to about 100 mg, about 25 mg to about 90 mg, about 25 mg to about 80 mg, about 25 mg to about 70 mg, about 25 mg to about 60 mg, about 25 mg to about 50 mg, about 25 mg to about 40 mg, about 25 mg to about 30 mg, about 50 mg to about 500 mg, about 50 mg to about 475 mg, about 50 mg to about 450 mg, about 50 mg to about 425 mg, about 50 mg to about 400 mg, about 50 mg to about 375 mg, about 50 mg to about 350 mg, about 50 mg to about 325 mg, about 50 mg to about 300 mg, about 50 mg to about 275 mg, about 50 mg to about 250 mg, about 50 mg to about 225 mg, about 50 mg to about 200 mg, about 50 mg to about 175 mg, about 50 mg to about 150 mg, about 50 mg to about 125 mg, about 50 mg to about 100 mg, about 50 mg to about 90 mg, about 50 mg to about 80 mg, about 50 mg to about 70 mg, about 50 mg to about 60 mg, about 75 mg to about 500 mg, about 75 mg to about 475 mg, about 75 mg to about 450 mg, about 75 mg to about 425 mg, about 75 mg to about 400 mg, about 75 mg to about 375 mg, about 75 mg to about 350 mg, about 75 mg to about 325 mg, about 75 mg to about 300 mg, about 75 mg to about 275 mg, about 75 mg to about 250 mg, about 75 mg to about 225 mg, about 75 mg to about 200 mg, about 75 mg to about 175 mg, about 75 mg to about 150 mg, about 75 mg to about 125 mg, about 75 mg to about 100 mg, about 75 mg to about 90 mg, about 75 mg to about 80 mg, about 100 mg to about 500 mg, about 100 mg to about 475 mg, about 100 mg to about 450 mg, about 100 mg to about 425 mg, about 100 mg to about 400 mg, about 100 mg to about 375 mg, about 100 mg to about 350 mg, about 100 mg to about 325 mg, about 100 mg to about 300 mg, about 100 mg to about 275 mg, about 100 mg to about 250 mg, about 100 mg to about 225 mg, about 100 mg to about 200 mg, about 100 mg to about 175 mg, about 100 mg to about 150 mg, about 100 mg to about 125 mg, about 150 mg to about 500 mg, about 150 mg to about 475 mg, about 150 mg to about 450 mg, about 150 mg to about 425 mg, about 150 mg to about 400 mg, about 150 mg to about 375 mg, about 150 mg to about 350 mg, about 150 mg to about 325 mg, about 150 mg to about 300 mg, about 150 mg to about 275 mg, about 150 mg to about 250 mg, about 150 mg to about 225 mg, about 150 mg to about 200 mg, about 150 mg to about 175 mg, about 200 mg to about 500 mg, about 200 mg to about 475 mg, about 200 mg to about 450 mg, about 200 mg to about 425 mg, about 200 mg to about 400 mg, about 200 mg to about 375 mg, about 200 mg to about 350 mg, about 200 mg to about 325 mg, about 200 mg to about 300 mg, about 200 mg to about 275 mg, about 200 mg to about 250 mg, about 200 mg to about 225 mg, about 250 mg to about 500 mg, about 250 mg to about 475 mg, about 250 mg to about 450 mg, about 250 mg to about 425 mg, about 250 mg to about 400 mg, about 250 mg to about 375 mg, about 250 mg to about 350 mg, about 250 mg to about 325 mg, about 250 mg to about 300 mg, about 250 mg to about 275 mg, about 300 mg to about 500 mg, about 300 mg to about 475 mg, about 300 mg to about 450 mg, about 300 mg to about 425 mg, about 300 mg to about 400 mg, about 300 mg to about 375 mg, about 300 mg to about 350 mg, about 300 mg to about 325 mg, about 350 mg to about 500 mg, about 350 mg to about 475 mg, about 350 mg to about 450 mg, about 350 mg to about 425 mg, about 350 mg to about 400 mg, about 350 mg to about 375 mg, about 400 mg to about 500 mg, about 400 mg to about 475 mg, about 400 mg to about 450 mg, about 400 mg to about 425 mg, about 35 mg, about 110 mg, about 160 mg, about 235 mg, about 360 mg or about 420 mg of magnesium.

In an embodiment, the composition comprises about 5 mg to about 50 mg of bitter melon extract. In an embodiment, the composition comprises about 5 mg to about 45 mg of bitter melon extract. In an embodiment, the composition comprises about 5 mg to about 40 mg of bitter melon extract. In further embodiments, the composition comprises about 5 mg to about 35 mg, 5 mg to about 30 mg, about 5 mg to about 25 mg, about 5 mg to about 20 mg, about 5 mg to about 15 mg, about 5 mg to about 10 mg, about 10 mg to about 50 mg, about 10 mg to about 45 mg, about 10 mg to about 40 mg, about 10 mg to about 35 mg, 10 mg to about 30 mg, about 10 mg to about 25 mg, about 10 mg to about 20 mg, about 10 mg to about 15 mg, about 15 mg to about 50 mg, about 15 mg to about 45 mg, about 15 mg to about 40 mg, about 15 mg to about 35 mg, 15 mg to about 30 mg, about 15 mg to about 25 mg, about 15 mg to about 20 mg, about 20 mg to about 50 mg, about 20 mg to about 45 mg, about 20 mg to about 40 mg, about 20 mg to about 35 mg, 20 mg to about 30 mg, about 20 mg to about 25 mg, about 25 mg to about 50 mg, about 25 mg to about 45 mg, about 25 mg to about 40 mg, about 25 mg to about 35 mg, 25 mg to about 30 mg, about 30 mg to about 50 mg, about 30 mg to about 45 mg, about 30 mg to about 40 mg, about 30 mg to about 35 mg, about 35 mg to about 50 mg, about 35 mg to about 45 mg, about 35 mg to about 40 mg, about 40 mg to about 50 mg, about 40 mg to about 45 mg, about 45 mg to about 50 mg, about 7 mg to about 13 mg, about 8 mg to about 12 mg, about 9 mg to about 11 mg, about 10 mg, about 11 mg, about 12 mg, about 13 mg, about 14 mg, about 15 mg, about 16 mg, about 17 mg, about 18 mg, about 28 mg or about 29 mg of bitter melon extract.

In an embodiment, the composition comprises about 5 mg to about 50 mg of fenugreek extract. In an embodiment, the composition comprises about 5 mg to about 45 mg of fenugreek extract. In an embodiment, the composition comprises about 5 mg to about 40 mg of fenugreek extract. In further embodiments, the composition comprises about 5 mg to about 35 mg, 5 mg to about 30 mg, about 5 mg to about 25 mg, about 5 mg to about 20 mg, about 5 mg to about 15 mg, about 5 mg to about 10 mg, about 10 mg to about 50 mg, about 10 mg to about 45 mg, about 10 mg to about 40 mg, about 10 mg to about 35 mg, 10 mg to about 30 mg, about 10 mg to about 25 mg, about 10 mg to about 20 mg, about 10 mg to about 15 mg, about 15 mg to about 50 mg, about 15 mg to about 45 mg, about 15 mg to about 40 mg, about 15 mg to about 35 mg, 15 mg to about 30 mg, about 15 mg to about 25 mg, about 15 mg to about 20 mg, about 20 mg to about 50 mg, about 20 mg to about 45 mg, about 20 mg to about 40 mg, about 20 mg to about 35 mg, 20 mg to about 30 mg, about 20 mg to about 25 mg, about 25 mg to about 50 mg, about 25 mg to about 45 mg, about 25 mg to about 40 mg, about 25 mg to about 35 mg, 25 mg to about 30 mg, about 30 mg to about 50 mg, about 30 mg to about 45 mg, about 30 mg to about 40 mg, about 30 mg to about 35 mg, about 35 mg to about 50 mg, about 35 mg to about 45 mg, about 35 mg to about 40 mg, about 40 mg to about 50 mg, about 40 mg to about 45 mg, about 45 mg to about 50 mg, about 7 mg to about 18 mg, about 8 mg to about 18 mg, or about 9 mg to about 22 mg of fenugreek extract

In an embodiment, the composition comprises about 50 mg to about 500 mg of the cinnamon extract. In an embodiment, the composition comprises about 50 mg to about 400 mg of the cinnamon extract. In an embodiment, the composition comprises about 50 mg to about 300 mg of the cinnamon extract. In further embodiment, the composition comprises about 50 mg to about 475 mg, about 50 mg to about 450 mg, about 50 mg to about 425 mg, about 50 mg to about 375 mg, about 50 mg to about 350 mg, about 50 mg to about 325 mg, about 50 mg to about 275 mg, about 50 mg to about 250 mg, about 50 mg to about 225 mg, about 50 mg to about 200 mg, about 50 mg to about 175 mg, about 50 mg to about 150 mg, about 50 mg to about 125 mg, 50 mg to about 100 mg, about 50 mg to about 75 mg, about 75 mg to about 500 mg, about 75 mg to about 475 mg, about 75 mg to about 450 mg, about 75 mg to about 425 mg, about 75 mg to about 400 mg, about 75 mg to about 375 mg, about 75 mg to about 350 mg, about 75 mg to about 325 mg, about 75 mg to about 300 mg, about 75 mg to about 275 mg, about 75 mg to about 250 mg, about 75 mg to about 225 mg, about 75 mg to about 200 mg, about 75 mg to about 175 mg, about 75 mg to about 150 mg, about 75 mg to about 125 mg, 75 mg to about 100 mg, about 100 mg to about 500 mg, about 100 mg to about 475 mg, about 100 mg to about 450 mg, about 100 mg to about 425 mg, about 100 mg to about 400 mg, about 100 mg to about 375 mg, about 100 mg to about 350 mg, about 100 mg to about 325 mg, about 100 mg to about 300 mg, about 100 mg to about 275 mg, about 100 mg to about 250 mg, about 100 mg to about 225 mg, about 100 mg to about 200 mg, about 100 mg to about 175 mg, about 100 mg to about 150 mg, about 100 mg to about 125 mg, about 150 mg to about 500 mg, about 150 mg to about 475 mg, about 150 mg to about 450 mg, about 150 mg to about 425 mg, about 150 mg to about 400 mg, about 150 mg to about 375 mg, about 150 mg to about 350 mg, about 150 mg to about 325 mg, about 150 mg to about 300 mg, about 150 mg to about 275 mg, about 150 mg to about 250 mg, about 150 mg to about 225 mg, about 150 mg to about 200 mg, about 150 mg to about 175 mg, about 200 mg to about 500 mg, about 200 mg to about 475 mg, about 200 mg to about 450 mg, about 200 mg to about 425 mg, about 200 mg to about 400 mg, about 200 mg to about 375 mg, about 200 mg to about 350 mg, about 200 mg to about 325 mg, about 200 mg to about 300 mg, about 200 mg to about 275 mg, about 200 mg to about 250 mg, about 200 mg to about 225 mg, about 225 mg to about 500 mg, about 225 mg to about 475 mg, about 225 mg to about 450 mg, about 225 mg to about 425 mg, about 225 mg to about 400 mg, about 225 mg to about 375 mg, about 225 mg to about 350 mg, about 225 mg to about 325 mg, about 225 mg to about 300 mg, about 225 mg to about 275 mg, about 225 mg to about 250 mg, about 250 mg to about 500 mg, about 250 mg to about 475 mg, about 250 mg to about 450 mg, about 250 mg to about 425 mg, about 250 mg to about 400 mg, about 250 mg to about 375 mg, about 250 mg to about 350 mg, about 250 mg to about 325 mg, about 250 mg to about 300 mg, about 250 mg to about 275 mg, about 275 mg to about 500 mg, about 275 mg to about 475 mg, about 275 mg to about 450 mg, about 275 mg to about 425 mg, about 275 mg to about 400 mg, about 275 mg to about 375 mg, about 275 mg to about 350 mg, about 275 mg to about 325 mg, about 275 mg to about 300 mg, about 300 mg to about 500 mg, about 300 mg to about 475 mg, about 300 mg to about 450 mg, about 300 mg to about 425 mg, about 300 mg to about 400 mg, about 300 mg to about 375 mg, about 300 mg to about 350 mg, about 300 mg to about 325 mg, about 325 mg to about 500 mg, about 325 mg to about 475 mg, about 325 mg to about 450 mg, about 325 mg to about 425 mg, about 325 mg to about 400 mg, about 325 mg to about 375 mg, about 325 mg to about 350 mg, about 350 mg to about 500 mg, about 350 mg to about 475 mg, about 350 mg to about 450 mg, about 350 mg to about 425 mg, about 350 mg to about 400 mg, about 350 mg to about 375 mg, about 400 mg to about 500 mg, about 400 mg to about 475 mg, about 400 mg to about 450 mg, about 400 mg to about 425 mg, about 425 mg to about 500 mg, about 425 mg to about 475 mg, about 425 mg to about 450 mg, about 450 mg to about 500 mg, about 450 mg to about 475 mg or about 475 mg to about 500 mg of the cinnamon extract.

In an embodiment, the composition comprises about 30 mg to about 150 mg of thiamine. In an embodiment, the composition comprises about 30 mg to about 140 mg of thiamine. In an embodiment, the composition comprises about 30 mg to about 130 mg of thiamine. In further embodiements, the composition comprises of about 30 mg to about 120 mg, about 30 mg to about 110 mg, about 30 mg to about 100 mg, about 30 mg to about 90 mg, about 30 mg to about 80 mg, about 30 mg to about 70 mg, about 30 mg to about 60 mg, about 30 mg to about 50 mg, about 30 mg to about 40 mg, about 40 mg to about 150 mg, about 40 mg to about 140 mg, about 40 mg to about 130 mg, about 40 mg to about 120 mg, about 40 mg to about 110 mg, about 40 mg to about 100 mg, about 40 mg to about 90 mg, about 40 mg to about 80 mg, about 40 mg to about 70 mg, about 40 mg to about 60 mg, about 40 mg to about 50 mg, about 50 mg to about 150 mg, about 50 mg to about 140 mg, about 50 mg to about 130 mg, about 50 mg to about 120 mg, about 50 mg to about 110 mg, about 50 mg to about 100 mg, about 50 mg to about 90 mg, about 50 mg to about 80 mg, about 50 mg to about 70 mg, about 50 mg to about 60 mg, about 60 mg to about 150 mg, about 60 mg to about 140 mg, about 60 mg to about 130 mg, about 60 mg to about 120 mg, about 60 mg to about 110 mg, about 60 mg to about 100 mg, about 60 mg to about 90 mg, about 60 mg to about 80 mg, about 60 mg to about 70 mg, about 70 mg to about 150 mg, about 70 mg to about 140 mg, about 70 mg to about 130 mg, about 70 mg to about 120 mg, about 70 mg to about 110 mg, about 70 mg to about 100 mg, about 70 mg to about 90 mg, about 70 mg to about 80 mg, about 80 mg to about 150 mg, about 80 mg to about 140 mg, about 80 mg to about 130 mg, about 80 mg to about 120 mg, about 80 mg to about 110 mg, about 80 mg to about 100 mg, about 80 mg to about 90 mg, about 90 mg to about 150 mg, about 90 mg to about 140 mg, about 90 mg to about 130 mg, about 90 mg to about 120 mg, about 90 mg to about 110 mg, about 90 mg to about 100 mg, about 100 mg to about 150 mg, about 100 mg to about 140 mg, about 100 mg to about 130 mg, about 100 mg to about 120 mg, about 100 mg to about 110 mg, about 110 mg to about 150 mg, about 110 mg to about 140 mg, about 110 mg to about 130 mg, about 110 mg to about 120 mg, about 120 mg to about 150 mg, about 120 mg to about 140 mg, about 120 mg to about 130 mg, about 130 mg to about 150 mg, about 130 mg to about 140 mg, about 140 mg to about 150 mg, about 75 mg, about 15 mg, about 55 mg, about 25 mg, about 115 mg or about 125 mg of thiamine

In an embodiment, the composition comprises about 25 mg to about 125 mg of ginger extract. In an embodiment, the composition comprises about 25 mg to about 120 mg of ginger extract. In an embodiment, the composition comprises about 25 mg to about 115 mg of ginger extract. In further embodiments, the composition comprises about 25 mg to about 110 mg, about 30 mg to about 125 mg, about 40 mg to about 120 mg, about 50 mg to about 125 mg, about 60 mg to about 125 mg, about 70 mg to about 125 mg, about 80 mg to about 125 mg, about 90 mg to about 125 mg, about 100 mg to about 125 mg, about 50 mg to about 120 mg, about 75 mg to about 120 mg, about 100 mg to about 120 mg, about 75 mg to about 115 mg, about 100 mg to about 115 mg, about 25 mg to about 100 mg, about 25 mg to about 80 mg, about 25 mg to about 60 mg, about 25 mg to about 50 mg, 25 mg to about 40 mg, about 25 mg to about 35 mg, about 30 mg to about 110 mg, about 30 mg to about 100 mg, about 30 mg to about 80 mg, about 30 mg to about 60 mg, about 30 mg to about 50 mg, 30 mg to about 40 mg, about 30 mg to about 35 mg, about 40 mg to about 110 mg, about 40 mg to about 100 mg, about 40 mg to about 80 mg, about 40 mg to about 60 mg, about 40 mg to about 50 mg, about 50 mg to about 110 mg, about 50 mg to about 100 mg, about 50 mg to about 80 mg, about 50 mg to about 60 mg, about 60 mg to about 110 mg, about 60 mg to about 100 mg, about 60 mg to about 80 mg, about 60 mg to about 75 mg, about 70 mg to about 110 mg, about 70 mg to about 100 mg, about 70 mg to about 80 mg, about 70 mg to about 75 mg, about 80 mg to about 110 mg, about 80 mg to about 100 mg, about 90 mg to about 110 mg, about 90 mg to about 100 mg, about 100 mg to about 110 mg or about 75 mg of ginger extract.

In an embodiment, the composition comprises about 5 mg to about 250 mg of ginseng. In an embodiment, the composition comprises about 5 mg to about 225 mg of ginseng. In an embodiment, the composition comprises about 5 mg to about 200 mg of ginseng. In further embodiment, the composition comprises about 5 mg to about 175 mg, about 5 mg to about 150 mg, about 5 mg to about 125 mg, about 5 mg to about 100 mg, about 5 mg to about 75 mg, about 5 mg to about 50 mg, about 5 mg to about 25 mg, about 25 mg to about 250 mg, about 25 mg to about 225 mg, about 25 mg to about 200 mg, about 25 mg to about 175 mg, about 25 mg to about 150 mg, about 25 mg to about 125 mg, about 25 mg to about 100 mg, about 25 mg to about 75 mg, about 25 mg to about 50 mg, about 50 mg to about 250 mg, about 50 mg to about 225 mg, about 50 mg to about 200 mg, about 50 mg to about 175 mg, about 50 mg to about 150 mg, about 50 mg to about 125 mg, about 50 mg to about 100 mg, about 50 mg to about 75 mg, about 75 mg to about 250 mg, about 75 mg to about 225 mg, about 75 mg to about 200 mg, about 75 mg to about 175 mg, about 75 mg to about 150 mg, about 75 mg to about 125 mg, about 75 mg to about 100 mg, about 85 mg to about 250 mg, about 85 mg to about 225 mg, about 85 mg to about 200 mg, about 85 mg to about 175 mg, about 85 mg to about 150 mg, about 85 mg to about 125 mg, about 85 mg to about 100 mg, about 95 mg to about 250 mg, about 95 mg to about 225 mg, about 95 mg to about 200 mg, about 95 mg to about 175 mg, about 95 mg to about 150 mg, about 95 mg to about 125 mg, about 95 mg to about 100 mg, about 105 mg to about 250 mg, about 105 mg to about 225 mg, about 105 mg to about 200 mg, about 105 mg to about 175 mg, about 105 mg to about 150 mg, about 105 mg to about 125 mg, about 115 mg to about 250 mg, about 115 mg to about 225 mg, about 115 mg to about 200 mg, about 115 mg to about 175 mg, about 115 mg to about 150 mg, about 115 mg to about 125 mg, about 125 mg to about 250 mg, about 125 mg to about 225 mg, about 125 mg to about 200 mg, about 125 mg to about 175 mg, about 125 mg to about 150 mg, about 135 mg to about 250 mg, about 135 mg to about 225 mg, about 135 mg to about 200 mg, about 135 mg to about 175 mg, about 135 mg to about 150 mg, about 145 mg to about 250 mg, about 145 mg to about 225 mg, about 145 mg to about 200 mg, about 145 mg to about 175 mg, about 145 mg to about 150 mg, about 155 mg to about 250 mg, about 155 mg to about 225 mg, about 155 mg to about 200 mg, about 155 mg to about 175 mg, about 165 mg to about 250 mg, about 165 mg to about 225 mg, about 165 mg to about 200 mg, about 165 mg to about 175 mg, about 175 mg to about 250 mg, about 175 mg to about 225 mg, about 175 mg to about 200 mg, about 185 mg to about 250 mg, about 185 mg to about 225 mg, about 185 mg to about 200 mg, about 195 mg to about 250 mg, about 195 mg to about 225 mg, about 195 mg to about 200 mg, about 205 mg to about 250 mg, about 205 mg to about 225 mg, about 215 mg to about 250 mg, about 215 mg to about 225 mg, about 225 mg to about 250 mg, about 235 mg to about 250 mg, about 245 mg to about 250 mg, about 7 mg to about 13 mg, about 8 mg to about 12 mg, about 9 mg to about 11 mg, or about 10 mg, of ginseng extract.

In an embodiment, the composition comprises about 0.5 mg to about 5.0 mg of copper. In an embodiment, the composition comprises about 0.5 mg to about 4.5 mg of copper. In further embodiments, the composition comprises about 0.5 mg to about 4.9 mg, about 0.5 mg to about 4.8 mg, about 0.5 mg to about 4.7 mg, about 0.5 mg to about 4.6 mg, about 0.5 mg to about 4.4 mg, about 0.5 mg to about 4.3 mg, about 0.5 mg to about 4.2 mg, about 0.5 mg to about 4.1 mg, about 0.5 mg to about 4.0 mg, about 0.5 mg to about 3.9 mg, about 0.5 mg to about 3.8 mg, about 0.5 mg to about 3.7 mg, about 0.5 mg to about 3.6 mg, about 0.5 mg to about 3.5 mg, about 0.5 mg to about 3.4 mg, about 0.5 mg to about 3.3 mg, about 0.5 mg to about 3.2 mg, about 0.5 mg to about 3.1 mg, about 0.5 mg to about 3.0 mg, about 0.5 mg to about 2.9 mg, about 0.5 mg to about 2.8 mg, about 0.5 mg to about 2.7 mg, about 0.5 mg to about 2.6 mg, about 0.5 mg to about 2.5 mg, about 0.5 mg to about 2.4 mg, about 0.5 mg to about 2.3 mg, about 0.5 mg to about 2.2 mg, about 0.5 mg to about 2.1 mg, about 0.5 mg to about 2.0 mg, about 0.5 mg to about 1.9 mg, about 0.5 mg to about 1.8 mg, about 0.5 mg to about 1.7 mg, about 0.5 mg to about 1.6 mg, about 0.5 mg to about 1.5 mg, about 0.5 mg to about 1.4 mg, about 0.5 mg to about 1.3 mg, about 0.5 mg to about 1.2 mg, about 0.5 mg to about 1.1 mg, about 0.5 mg to about 0.9 mg, about 0.5 mg to about 0.8 mg, about 0.5 mg to about 0.7 mg, about 0.5 mg to about 0.6 mg, about 0.75 mg to about 5.0 mg, about 0.75 mg to about 4.5 mg, 0.75 mg to about 4.9 mg, about 0.75 mg to about 4.8 mg, about 0.75 mg to about 4.7 mg, about 0.75 mg to about 4.6 mg, about 0.75 mg to about 4.4 mg, about 0.75 mg to about 4.3 mg, about 0.75 mg to about 4.2 mg, about 0.75 mg to about 4.1 mg, about 0.75 mg to about 4.0 mg, about 0.75 mg to about 3.9 mg, about 0.75 mg to about 3.8 mg, about 0.75 mg to about 3.7 mg, about 0.75 mg to about 3.6 mg, about 0.75 mg to about 3.5 mg, about 0.75 mg to about 3.4 mg, about 0.75 mg to about 3.3 mg, about 0.75 mg to about 3.2 mg, about 0.75 mg to about 3.1 mg, about 0.75 mg to about 3.0 mg, about 0.75 mg to about 2.9 mg, about 0.75 mg to about 2.8 mg, about 0.75 mg to about 2.7 mg, about 0.75 mg to about 2.6 mg, about 0.75 mg to about 2.5 mg, about 0.75 mg to about 2.4 mg, about 0.75 mg to about 2.3 mg, about 0.75 mg to about 2.2 mg, about 0.75 mg to about 2.1 mg, about 0.75 mg to about 2.0 mg, about 0.75 mg to about 1.9 mg, about 0.75 mg to about 1.8 mg, about 0.75 mg to about 1.7 mg, about 0.75 mg to about 1.6 mg, about 0.75 mg to about 1.5 mg, about 0.75 mg to about 1.4 mg, about 0.75 mg to about 1.3 mg, about 0.75 mg to about 1.2 mg, about 0.75 mg to about 1.1 mg, about 0.75 mg to about 0.9 mg, about 0.75 mg to about 0.8 mg, about 1.0 mg to about 5.0 mg, about 1.0 mg to about 4.5 mg, 1.0 mg to about 4.9 mg, about 1.0 mg to about 4.8 mg, about 1.0 mg to about 4.7 mg, about 1.0 mg to about 4.6 mg, about 1.0 mg to about 4.4 mg, about 1.0 mg to about 4.3 mg, about 1.0 mg to about 4.2 mg, about 1.0 mg to about 4.1 mg, about 1.0 mg to about 4.0 mg, about 1.0 mg to about 3.9 mg, about 1.0 mg to about 3.8 mg, about 1.0 mg to about 3.7 mg, about 1.0 mg to about 3.6 mg, about 1.0 mg to about 3.5 mg, about 1.0 mg to about 3.4 mg, about 1.0 mg to about 3.3 mg, about 1.0 mg to about 3.2 mg, about 1.0 mg to about 3.1 mg, about 1.0 mg to about 3.0 mg, about 1.0 mg to about 2.9 mg, about 1.0 mg to about 2.8 mg, about 1.0 mg to about 2.7 mg, about 1.0 mg to about 2.6 mg, about 1.0 mg to about 2.5 mg, about 1.0 mg to about 2.4 mg, about 1.0 mg to about 2.3 mg, about 1.0 mg to about 2.2 mg, about 1.0 mg to about 2.1 mg, about 1.0 mg to about 2.0 mg, about 1.0 mg to about 1.9 mg, about 1.0 mg to about 1.8 mg, about 1.0 mg to about 1.7 mg, about 1.0 mg to about 1.6 mg, about 1.0 mg to about 1.5 mg, about 1.0 mg to about 1.4 mg, about 1.0 mg to about 1.3 mg, about 1.0 mg to about 1.2 mg, about 1.0 mg to about 1.1 mg, about 1.5 mg to about 5.0 mg, about 1.5 mg to about 4.5 mg, 1.5 mg to about 4.9 mg, about 1.5 mg to about 4.8 mg, about 1.5 mg to about 4.7 mg, about 1.5 mg to about 4.6 mg, about 1.5 mg to about 4.4 mg, about 1.5 mg to about 4.3 mg, about 1.5 mg to about 4.2 mg, about 1.5 mg to about 4.1 mg, about 1.5 mg to about 4.0 mg, about 1.5 mg to about 3.9 mg, about 1.5 mg to about 3.8 mg, about 1.5 mg to about 3.7 mg, about 1.5 mg to about 3.6 mg, about 1.5 mg to about 3.5 mg, about 1.5 mg to about 3.4 mg, about 1.5 mg to about 3.3 mg, about 1.5 mg to about 3.2 mg, about 1.5 mg to about 3.1 mg, about 1.5 mg to about 3.0 mg, about 1.5 mg to about 2.9 mg, about 1.5 mg to about 2.8 mg, about 1.5 mg to about 2.7 mg, about 1.5 mg to about 2.6 mg, about 1.5 mg to about 2.5 mg, about 1.5 mg to about 2.4 mg, about 1.5 mg to about 2.3 mg, about 1.5 mg to about 2.2 mg, about 1.5 mg to about 2.1 mg, about 1.5 mg to about 2.0 mg, about 1.5 mg to about 1.9 mg, about 1.5 mg to about 1.8 mg, about 1.5 mg to about 1.7 mg, about 1.5 mg to about 1.6 mg, about 2.0 mg to about 5.0 mg, about 2.0 mg to about 4.5 mg, 2.0 mg to about 4.9 mg, about 2.0 mg to about 4.8 mg, about 2.0 mg to about 4.7 mg, about 2.0 mg to about 4.6 mg, about 2.0 mg to about 4.4 mg, about 2.0 mg to about 4.3 mg, about 2.0 mg to about 4.2 mg, about 2.0 mg to about 4.1 mg, about 2.0 mg to about 4.0 mg, about 2.0 mg to about 3.9 mg, about 2.0 mg to about 3.8 mg, about 2.0 mg to about 3.7 mg, about 2.0 mg to about 3.6 mg, about 2.0 mg to about 3.5 mg, about 2.0 mg to about 3.4 mg, about 2.0 mg to about 3.3 mg, about 2.0 mg to about 3.2 mg, about 2.0 mg to about 3.1 mg, about 2.0 mg to about 3.0 mg, about 2.0 mg to about 2.9 mg, about 2.0 mg to about 2.8 mg, about 2.0 mg to about 2.7 mg, about 2.0 mg to about 2.6 mg, about 2.0 mg to about 2.5 mg, about 2.0 mg to about 2.4 mg, about 2.0 mg to about 2.3 mg, about 2.0 mg to about 2.2 mg, about 2.0 mg to about 2.1 mg, about 2.5 mg to about 5.0 mg, about 2.5 mg to about 4.5 mg, 2.5 mg to about 4.9 mg, about 2.5 mg to about 4.8 mg, about 2.5 mg to about 4.7 mg, about 2.5 mg to about 4.6 mg, about 2.5 mg to about 4.4 mg, about 2.5 mg to about 4.3 mg, about 2.5 mg to about 4.2 mg, about 2.5 mg to about 4.1 mg, about 2.5 mg to about 4.0 mg, about 2.5 mg to about 3.9 mg, about 2.5 mg to about 3.8 mg, about 2.5 mg to about 3.7 mg, about 2.5 mg to about 3.6 mg, about 2.5 mg to about 3.5 mg, about 2.5 mg to about 3.4 mg, about 2.5 mg to about 3.3 mg, about 2.5 mg to about 3.2 mg, about 2.5 mg to about 3.1 mg, about 2.5 mg to about 3.0 mg, about 2.5 mg to about 2.9 mg, about 2.5 mg to about 2.8 mg, about 2.5 mg to about 2.7 mg, about 2.5 mg to about 2.6 mg, about 3.0 mg to about 5.0 mg, about 3.0 mg to about 4.5 mg, 3.0 mg to about 4.9 mg, about 3.0 mg to about 4.8 mg, about 3.0 mg to about 4.7 mg, about 3.0 mg to about 4.6 mg, about 3.0 mg to about 4.4 mg, about 3.0 mg to about 4.3 mg, about 3.0 mg to about 4.2 mg, about 3.0 mg to about 4.1 mg, about 3.0 mg to about 4.0 mg, about 3.0 mg to about 3.9 mg, about 3.0 mg to about 3.8 mg, about 3.0 mg to about 3.7 mg, about 3.0 mg to about 3.6 mg, about 3.0 mg to about 3.5 mg, about 3.0 mg to about 3.4 mg, about 3.0 mg to about 3.3 mg, about 3.0 mg to about 3.2 mg, about 3.0 mg to about 3.1 mg, about 3.5 mg to about 5.0 mg, about 3.5 mg to about 4.5 mg, 3.5 mg to about 4.9 mg, about 3.5 mg to about 4.8 mg, about 3.5 mg to about 4.7 mg, about 3.5 mg to about 4.6 mg, about 3.5 mg to about 4.4 mg, about 3.5 mg to about 4.3 mg, about 3.5 mg to about 4.2 mg, about 3.5 mg to about 4.1 mg, about 3.5 mg to about 4.0 mg, about 3.5 mg to about 3.9 mg, about 3.5 mg to about 3.8 mg, about 3.5 mg to about 3.7 mg, about 3.5 mg to about 3.6 mg, about 4.0 mg to about 5.0 mg, about 4.0 mg to about 4.5 mg, 4.0 mg to about 4.9 mg, about 4.0 mg to about 4.8 mg, about 4.0 mg to about 4.7 mg, about 4.0 mg to about 4.6 mg, about 4.0 mg to about 4.4 mg, about 4.0 mg to about 4.3 mg, about 4.0 mg to about 4.2 mg, about 4.0 mg to about 4.1 mg, about 4.5 mg to about 5.0 mg, about 4.5 mg to about 4.5 mg, 4.5 mg to about 4.9 mg, about 4.5 mg to about 4.8 mg, about 4.5 mg to about 4.7 mg, about 4.5 mg to about 4.6 mg, about 0.7 mg to about 1.3 mg, about 0.8 mg to about 1.2 mg, about 0.9 mg to about 1.1 mg, about 2 mg or about 1 mg of copper.

In an embodiment, the composition comprises the following ingredients:
(a) 5% to 25% (w/w) of coenzyme Q10;
(b) 0.01% to 5.0% (w/w) of chromium;
(c) 5% to 15% (w/w) of zinc;
(d) 4% to 15% (w/w) of magnesium;
(e) 1% to 15% (w/w) of bitter melon extract;
(f) 1% to 15% (w/w) of fenugreek extract;
(g) 20% to 70% (w/w) of cinnamon extract;
(h) 10% to 25% (w/w) of thiamine;
(i) 10% to 30% (w/w) of ginger extract;
(j) 1% to 20% (w/w) of ginseng extract; and
(k) 0.1% to 5.0% (w/w) of copper.

In an embodiment, the composition comprises the following ingredients:
(a) 5% to 20% (w/w) of coenzyme Q10;
(b) 0.5% to 5.0% (w/w) of chromium;
(c) 5% to 10% (w/w) of zinc;
(d) 4% to 10% (w/w) of magnesium;
(e) 1% to 10% (w/w) of bitter melon extract;
(f) 1% to 10% (w/w) of fenugreek extract;
(g) 20% to 50% (w/w) of cinnamon extract;
(h) 10% to 20% (w/w) of thiamine;
(i) 10% to 20% (w/w) of ginger extract;
(j) 1% to 15% (w/w) of ginseng extract; and
(k) 0.5% to 5.0% (w/w) of copper.

In an embodiment, the composition comprises the following ingredients:
(a) 5% to 15% (w/w) of coenzyme Q10;
(b) 0.5% to 4.0% (w/w) of chromium;
(c) 5% to 8% (w/w) of zinc;
(d) 4% to 8% (w/w) of magnesium;
(e) 1% to 8% (w/w) of bitter melon extract;
(f) 1% to 8% (w/w) of fenugreek extract;
(g) 20% to 40% (w/w) of cinnamon extract;
(h) 10% to 16% (w/w) of thiamine;
(i) 10% to 15% (w/w) of ginger extract;
(j) 1% to 10% (w/w) of ginseng extract; and
(k) 0.5% to 4.0% (w/w) of copper.

In an embodiment, the composition comprises of the following ingredients:
(a) 10% to 15% (w/w) of coenzyme Q10;
(b) 1.0% to 4.0% (w/w) of chromium;
(c) 6% to 10% (w/w) of zinc;
(d) 8% to 12% (w/w) of magnesium;
(e) 4% to 10% (w/w) of bitter melon extract;
(f) 4% to 10% (w/w) of fenugreek extract;
(g) 30% to 50% (w/w) of cinnamon extract;
(h) 15% to 18% (w/w) of thiamine;
(i) 12% to 15% (w/w) of ginger extract;
(j) 2% to 10% (w/w) of ginseng extract; and
(k) 1.0% to 4.0% (w/w) of copper.

In an embodiment, the composition comprises the following ingredients:
(a) 30 mg to 120 mg of coenzyme Q10;
(b) 0.1 mg to 0.8 mg of chromium;
(c) 20 mg to 100 mg of zinc;
(d) 10 mg to 500 mg of magnesium;
(e) 5 mg to 50 mg of bitter melon extract;
(f) 5 mg to 50 mg of fenugreek extract;
(g) 50 mg to 500 mg of cinnamon extract;
(h) 30 mg to 150 mg of thiamine;
(i) 25 mg to 125 mg of ginger extract;
(j) 5 mg to 250 mg of ginseng extract; and
(k) 0.5 mg to 5.0 mg of copper.

In an embodiment, the composition comprises the following ingredients:
(a) 30 mg to 100 mg of coenzyme Q10;
(b) 0.1 mg to 0.6 mg of chromium;
(c) 20 mg to 75 mg of zinc;
(d) 10 mg to 400 mg of magnesium;
(e) 5 mg to 40 mg of bitter melon extract;
(f) 5 mg to 40 mg of fenugreek extract;
(g) 50 mg to 400 mg of cinnamon extract;
(h) 30 mg to 100 mg of thiamine;
(i) 25 mg to 100 mg of ginger extract;
(j) 5 mg to 200 mg of ginseng extract; and
(k) 0.5 mg to 4.0 mg of copper.

In an embodiment, the composition comprises the following ingredients:
(a) 50 mg to 100 mg of coenzyme Q10;
(b) 0.4 mg to 0.6 mg of chromium;
(c) 20 mg to 75 mg of zinc;
(d) 50 mg to 400 mg of magnesium;
(e) 10 mg to 40 mg of bitter melon extract;
(f) 10 mg to 40 mg of fenugreek extract;
(g) 100 mg to 400 mg of cinnamon extract;
(h) 50 mg to 100 mg of thiamine;
(i) 50 mg to 100 mg of ginger extract;
(j) 50 mg to 200 mg of ginseng extract; and
(k) 1.0 mg to 4.0 mg of copper.

In an embodiment, the composition comprises the following ingredients:
(a) 50 mg to 75 mg of coenzyme Q10;
(b) 0.4 mg to 0.8 mg of chromium;
(c) 20 mg to 50 mg of zinc;
(d) 50 mg to 200 mg of magnesium;
(e) 10 mg to 20 mg of bitter melon extract;
(f) 10 mg to 20 mg of fenugreek extract;
(g) 100 mg to 300 mg of cinnamon extract;
(h) 50 mg to 75 mg of thiamine;
(i) 50 mg to 75 mg of ginger extract;
(j) 50 mg to 100 mg of ginseng extract; and
(k) 1.0 mg to 3.0 mg of copper.

In an embodiment, the composition of each dose comprises the following ingredients: (a) 50 mg of coenzyme Q10; (b) 250 mcg of of chromium; (c) 40 mg of zinc; (d) 150 mg of magnesium; (e) 10 mg of bitter melon extract; (f) 10 mg of fenugreek extract; (g) 100 mg of cinnamon extract; (h) 50 mg of thiamine; (i) 55 mg of ginger extract; (j) 10 mg of ginseng extract; and (k) 1.0 mg of copper.

In an embodiment, the composition of each dose comprises the following ingredients: (a) 50 mg of coenzyme Q10; (b) 250 mcg of of chromium; (c) 40 mg of zinc; (d) 30 mg of magnesium; (e) 10 mg of bitter melon extract; (f) 10 mg of fenugreek extract; (g) 100 mg of cinnamon extract; (h) 50 mg of thiamine; (i) 55 mg of ginger extract; (j) 10 mg of ginseng extract; and (k) 1.0 mg of copper.

In an embodiment, the composition of each dose comprises the following ingredients: (a) 100 mg of coenzyme Q10; (b) 500 mcg of of chromium; (c) 80 mg of zinc; (d) 300 mg of magnesium; (e) 20 mg of bitter melon extract; (f) 20 mg of fenugreek extract; (g) 200 mg of cinnamon extract; (h) 100 mg of thiamine; (i) 110 mg of ginger extract; (j) 40 mg of ginseng extract; and (k) 2.0 mg of copper. In an embodiment, the composition of each dose comprises the following ingredients: (a) 100 mg of coenzyme Q10; (b) 500 mcg of of chromium; (c) 80 mg of zinc; (d) 60 mg of magnesium; (e) 20 mg of bitter melon extract; (f) 20 mg of fenugreek extract; (g) 200 mg of cinnamon extract; (h) 100 mg of thiamine; (i) 110 mg of ginger extract; (j) 20 mg of ginseng extract; and (k) 2.0 mg of copper. In another embodiment, the composition further comprises at least one carrier or excipient, in a further embodiment a pharmaceutically or nutritionally acceptable carrier or excipient. Such compositions may be prepared in a manner well known in the pharmaceutical art (see Remington: The Science and Practice of Pharmacy, by Loyd V Allen, Jr, 2012, 22nd edition, Pharmaceutical Press; Handbook of Pharmaceutical Excipients, by Rowe et al., 2012, 7th edition, Pharmaceutical Press).

An “excipient,” as used herein, has its normal meaning in the art and is any ingredient that is not an active ingredient (drug) itself. Excipients include for example binders, lubricants, diluents, fillers, thickening agents, disintegrants, plasticizers, coatings, barrier layer formulations, lubricants, stabilizing agent, release-delaying agents and other components. “Pharmaceutically acceptable excipient” or “nutritionally acceptable excipient” as used herein refers to any excipient that does not interfere with effectiveness of the biological activity of the active ingredients and that is not toxic to the subject, i.e., is a type of excipient and/or is for use in an amount which is not toxic to the subject. Excipients are well known in the art, and the present system is not limited in these respects. In certain embodiments, the composition includes excipients, including for example and without limitation, one or more binders (binding agents), thickening agents, surfactants, diluents, release-delaying agents, colorants, flavoring agents, fillers, disintegrants/dissolution promoting agents, lubricants, plasticizers, silica flow conditioners, glidants, anti-caking agents, anti-tacking agents, stabilizing agents, anti-static agents, swelling agents and any combinations thereof. As those of skill would recognize, a single excipient can fulfill more than two functions at once, e.g., can act as both a binding agent and a thickening agent. As those of skill will also recognize, these terms are not necessarily mutually exclusive.

The excipient may be selected for administration of the composition by various administration routes, for example for oral administration, subcutaneous administration, intradermal or transdermal administration, or parenteral administration.

Useful diluents, e.g., fillers, include, for example and without limitation, dicalcium phosphate, calcium diphosphate, calcium carbonate, calcium sulfate, lactose, cellulose, kaolin, sodium chloride, starches, powdered sugar, colloidal silicon dioxide, titanium oxide, alumina, talc, colloidal silica, microcrystalline cellulose, silicified micro crystalline cellulose and combinations thereof. Fillers that can add bulk to tablets with minimal drug dosage to produce tablets of adequate size and weight include croscarmellose sodium NF/EP (e.g., Ac-Di-Sol); anhydrous lactose NF/EP (e.g., Pharmatose™ DCL 21); and/or povidone USP/EP.

Binder materials include, for example and without limitation, starches (including corn starch and pregelatinized starch), gelatin, sugars (including sucrose, glucose, dextrose and lactose), polyethylene glycol, povidone, waxes, and natural and synthetic gums, e.g., acacia sodium alginate, polyvinylpyrrolidone, cellulosic polymers (e.g., hydroxypropyl cellulose, hydroxypropyl methylcellulose, methyl cellulose, hydroxyethyl cellulose, carboxymethylcellulose, colloidal silicon dioxide NF/EP (e.g., Cab-O-Sil™ M5P), Silicified Microcrystalline Cellulose (SMCC), e.g., Silicified microcrystalline cellulose NF/EP (e.g., Prosolv™ SMCC 90, COMPRECEL®), and silicon dioxide, mixtures thereof, and the like), veegum, and combinations thereof. In an embodiment, the composition or dosage form described herein comprises gelatin and/or microcrystalline cellulose.

Useful lubricants include, for example, canola oil, glyceryl palmitostearate, hydrogenated vegetable oil (type I), magnesium oxide, magnesium stearate, mineral oil, poloxamer, polyethylene glycol, sodium lauryl sulfate, sodium stearate fumarate, stearic acid, talc and, zinc stearate, glyceryl behapate, magnesium lauryl sulfate, boric acid, sodium benzoate, sodium acetate, sodium benzoate/sodium acetate (in combination), DL-leucine, calcium stearate, sodium stearyl fumarate, mixtures thereof, and the like. In an embodiment, the composition or dosage form described herein comprises magnesium stearate.

Bulking agents include, for example: microcrystalline cellulose, for example, AVICEL® (FMC Corp.), COMPRECEL®, EMCOCEL® (Mendell Inc.), which also has binder properties; dicalcium phosphate, for example, EMCOMPRESS® (Mendell Inc.); calcium sulfate, for example, COMPACTROL® (Mendell Inc.); and starches, for example, Starch 1500; and polyethylene glycols (CARBOWAX®).

Disintegrating or dissolution promoting agents include: starches, clays, celluloses, alginates, gums, crosslinked polymers, colloidal silicon dioxide, osmogens, mixtures thereof, and the like, such as crosslinked sodium carboxymethyl cellulose (AC-DI-SOL®), sodium croscarmelose, sodium starch glycolate (EXPLOTAB®, PRIMO JEL®) crosslinked polyvinylpolypyrrolidone (PLASONE-XL®), sodium chloride, sucrose, lactose and mannitol.

Antiadherents and glidants employable in the core and/or a coating of the solid oral dosage form may include talc, starches (e.g., cornstarch), celluloses, silicon dioxide, sodium lauryl sulfate, colloidal silica dioxide, and metallic stearates, among others.

Examples of silica flow conditioners include colloidal silicon dioxide, magnesium aluminum silicate and guar gum.

Suitable surfactants include pharmaceutically acceptable non-ionic, ionic and anionic surfactants. An example of a surfactant is sodium lauryl sulfate. If desired, the pharmaceutical composition to be administered may also contain minor amounts of nontoxic auxiliary substances such as wetting or emulsifying agents, pH-buffering agents and the like, for example, sodium acetate, sorbitan monolaurate, triethanolamine sodium acetate, triethanolamine oleate, etc. If desired, flavoring, coloring and/or sweetening agents may be added as well.

Examples of stabilizing agents include acacia, albumin, polyvinyl alcohol, alginic acid, bentonite, dicalcium phosphate, carboxymethylcellulose, hydroxypropylcellulose, colloidal silicon dioxide, cyclodextrins, glyceryl monostearate, hydroxypropyl methylcellulose, magnesium trisilicate, magnesium aluminum silicate, propylene glycol, propylene glycol alginate, sodium alginate, carnauba wax, xanthan gum, starch, stearate(s), stearic acid, stearic monoglyceride and stearyl alcohol.

Examples of thickening agent can be for example talc USP/EP, a natural gum, such as guar gum, or a cellulose derivative such as microcrystalline cellulose NF/EP (e.g., Avicel™ PH 102), methylcellulose, ethylcellulose or hydroxyethylcellulose. A useful thickening agent is hydroxypropyl methylcellulose, an adjuvant which is available in various viscosity grades.

Examples of plasticizers include: acetylated monoglycerides; these can be used as food additives; Alkyl citrates, used in food packagings, medical products, cosmetics and children toys; Triethyl citrate (TEC); Acetyl triethyl citrate (ATEC), higher boiling point and lower volatility than TEC; Tributyl citrate (TBC); Acetyl tributyl citrate (ATBC), compatible with PVC and vinyl chloride copolymers; Trioctyl citrate (TOC), also used for gums and controlled release medicines; Acetyl trioctyl citrate (ATOC), also used for printing ink; Trihexyl citrate (THC), compatible with PVC, also used for controlled release medicines; Acetyl trihexyl citrate (ATHC), compatible with PVC; Butyryl trihexyl citrate (BTHC, trihexyl o-butyryl citrate), compatible with PVC; Trimethyl citrate (TMC), compatible with PVC; alkyl sulphonic acid phenyl ester, polyethylene glycol (PEG) or any combination thereof. Optionally, the plasticizer can comprise triethyl citrate NF/EP.

Examples of permeation enhancers include: sulphoxides (such as dimethylsulphoxide, DMSO), azones (e.g., laurocapram), pyrrolidones (for example 2-pyrrolidone, 2P), alcohols and alkanols (ethanol, or decanol), glycols (for example propylene glycol and polyethylene glycol), surfactants and terpenes.

In an embodiment, the composition is formulated for oral administration, e.g., is an oral dosage form. Formulations suitable for oral administration may include (a) liquid solutions, such as an effective amount of active agent(s)/composition(s) suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient(s), carriers known in the art.

In an embodiment, the composition described herein is encapsulated. Thus, in another embodiment, the present disclosure provides a capsule comprising the composition described herein. In an embodiment, the capsule is a hard-shelled capsule. The capsule may be made of any material commonly used in the drug and/or food industry. Example of materials suitable for capsules include gelling agents, such as animal protein (mainly gelatin) or plant polysaccharides or their derivatives (such as carrageenan and modified forms of starch and cellulose such as hypromellose). Other ingredients can be added to the gelling agent including plasticizers such as glycerin or sorbitol to decrease the capsule's hardness, coloring agents, preservatives, disintegrants and/or lubricants. In an embodiment, the capsule is a hard gelatin capsule. The capsule may be of any size (i.e., size #000 to #5), which is selected according to the desired amount of composition to be encapsulated. In an embodiment, the size of the capsule is preferably size #000 to #0, for example size #00. The process of encapsulation of hard capsules such as hard gelatin capsules may be done on manual, semi-automatic and automatic apparatuses, which are well known in the art.

In an embodiment, the composition described herein is in a liquid formulation. In an embodiment, the composition described herein is in a liquid solution. In an embodiment, the composition described herein is in a liquid solution, such as an effective amount of active agent(s)/composition(s) suspended in diluents, such as water, saline or PEG 400. In an embodiment, the composition described herein is in the form of sachets containing a predetermined amount of the active ingredient, as liquids, solids, granules or gelatin. In an embodiment, the composition described herein is in the form of tablets. In an embodiment, the composition described herein is in the form of granules. In an embodiment, the composition described herein is in the form of suspensions in an appropriate liquid. In an embodiment, the composition described herein is in the form of a dry powder to be dissolved in a liquid. In an embodiment, the composition described herein is in the form of a disintegrating tablets. In an embodiment, the composition described herein is in the form of a syrup.

In an embodiment, the composition described herein is in the form of a liquid administered as drops. In an embodiment, the composition described herein is in the form of chewable tablets. In an embodiment, the composition described herein is in the form of lozenges. In an embodiment, the composition described herein is in the form of effervescent tablets. In an embodiment, the composition described herein is in the form of effervescent dry powder.

Thus, in another aspect, the present disclosure provides a dosage form, such as an oral dosage form, comprising the composition described herein. The term “dosage form” as used herein refers to a product, such as a pharmaceutical drug product, in the form in which it is marketed for use, i.e., in a particular configuration (e.g., capsule shell, tablet), and apportioned into a particular dose. In an embodiment, the dosage form is an oral dosage form, for example a capsule.

The dosage form may comprise any suitable amount of the composition described herein. In an embodiment, the dosage form comprises from about 100 mg to about 2000 mg of the composition described herein. In further embodiments from about 100 mg to about 2500 mg, from about 100 mg to about 2400 mg, from about 100 mg to about 2300 mg, from about 100 mg to about 2200 mg, from about 100 mg to about 2100 mg, from about 100 mg to about 1900 mg, from about 100 mg to about 1800 mg, from about 100 mg to about 1700 mg, from about 100 mg to about 1600 mg, from about 100 mg to about 1500 mg, from about 100 mg to about 1400 mg, from about 100 mg to about 1300 mg, from about 100 mg to about 1200 mg, from about 100 mg to about 1100 mg, from about 100 mg to about 1000 mg, from about 100 mg to about 900 mg, from about 100 mg to about 800 mg, from about 100 mg to about 700 mg, from about 100 mg to about 600 mg, from about 100 mg to about 500 mg, from about 100 mg to about 400 mg, from about 100 mg to about 300 mg, from about 100 mg to about 200 mg, from about 200 mg to about 2500 mg, from about 200 mg to about 2400 mg, from about 200 mg to about 2300 mg, from about 200 mg to about 2200 mg, from about 200 mg to about 2100 mg, from about 200 mg to about 1900 mg, from about 200 mg to about 2000 mg, from about 200 mg to about 1800 mg, from about 200 mg to about 1700 mg, from about 200 mg to about 1600 mg, from about 200 mg to about 1500 mg, from about 200 mg to about 1400 mg, from about 200 mg to about 1300 mg, from about 200 mg to about 1200 mg, from about 200 mg to about 1100 mg, from about 200 mg to about 1000 mg, from about 200 mg to about 900 mg, from about 200 mg to about 800 mg, from about 200 mg to about 700 mg, from about 200 mg to about 600 mg, from about 200 mg to about 500 mg, from about 200 mg to about 400 mg, from about 200 mg to about 300 mg, from about 300 mg to about 2500 mg, from about 300 mg to about 2400 mg, from about 300 mg to about 2300 mg, from about 300 mg to about 2200 mg, from about 300 mg to about 2100 mg, from about 300 mg to about 1900 mg, from about 300 mg to about 2000 mg, from about 300 mg to about 1800 mg, from about 300 mg to about 1700 mg, from about 300 mg to about 1600 mg, from about 300 mg to about 1500 mg, from about 300 mg to about 1400 mg, from about 300 mg to about 1300 mg, from about 300 mg to about 1200 mg, from about 300 mg to about 1100 mg, from about 300 mg to about 1000 mg, from about 300 mg to about 900 mg, from about 300 mg to about 800 mg, from about 300 mg to about 700 mg, from about 300 mg to about 600 mg, from about 300 mg to about 500 mg, from about 300 mg to about 400 mg, from about 400 mg to about 2500 mg, from about 400 mg to about 2400 mg, from about 400 mg to about 2300 mg, from about 400 mg to about 2200 mg, from about 400 mg to about 2100 mg, from about 400 mg to about 1900 mg, from about 400 mg to about 2000 mg, from about 400 mg to about 1800 mg, from about 400 mg to about 1700 mg, from about 400 mg to about 1600 mg, from about 400 mg to about 1500 mg, from about 400 mg to about 1400 mg, from about 400 mg to about 1300 mg, from about 400 mg to about 1200 mg, from about 400 mg to about 1100 mg, from about 400 mg to about 1000 mg, from about 400 mg to about 900 mg, from about 400 mg to about 800 mg, from about 400 mg to about 700 mg, from about 400 mg to about 600 mg, from about 400 mg to about 500 mg, from about 500 mg to about 2500 mg, from about 500 mg to about 2400 mg, from about 500 mg to about 2300 mg, from about 500 mg to about 2200 mg, from about 500 mg to about 2100 mg, from about 500 mg to about 1900 mg, from about 500 mg to about 2000 mg, from about 500 mg to about 1800 mg, from about 500 mg to about 1700 mg, from about 500 mg to about 1600 mg, from about 500 mg to about 1500 mg, from about 500 mg to about 1400 mg, from about 500 mg to about 1300 mg, from about 500 mg to about 1200 mg, from about 500 mg to about 1100 mg, from about 500 mg to about 1000 mg, from about 500 mg to about 900 mg, from about 500 mg to about 800 mg, from about 500 mg to about 700 mg, from about 500 mg to about 600 mg, from about 600 mg to about 2500 mg, from about 600 mg to about 2400 mg, from about 600 mg to about 2300 mg, from about 600 mg to about 2200 mg, from about 600 mg to about 2100 mg, from about 600 mg to about 1900 mg, from about 600 mg to about 2000 mg, from about 600 mg to about 1800 mg, from about 600 mg to about 1700 mg, from about 600 mg to about 1600 mg, from about 600 mg to about 1500 mg, from about 600 mg to about 1400 mg, from about 600 mg to about 1300 mg, from about 600 mg to about 1200 mg, from about 600 mg to about 1100 mg, from about 600 mg to about 1000 mg, from about 600 mg to about 900 mg, from about 600 mg to about 800 mg, from about 600 mg to about 700 mg, from about 700 mg to about 2500 mg, from about 700 mg to about 2400 mg, from about 700 mg to about 2300 mg, from about 700 mg to about 2200 mg, from about 700 mg to about 2100 mg, from about 700 mg to about 1900 mg, from about 700 mg to about 2000 mg, from about 700 mg to about 1800 mg, from about 700 mg to about 1700 mg, from about 700 mg to about 1600 mg, from about 700 mg to about 1500 mg, from about 700 mg to about 1400 mg, from about 700 mg to about 1300 mg, from about 700 mg to about 1200 mg, from about 700 mg to about 1100 mg, from about 700 mg to about 1000 mg, from about 700 mg to about 900 mg, from about 700 mg to about 800 mg, from about 900 mg to about 2500 mg, from about 900 mg to about 2400 mg, from about 900 mg to about 2300 mg, from about 900 mg to about 2200 mg, from about 900 mg to about 2100 mg, from about 900 mg to about 1900 mg, from about 900 mg to about 2000 mg, from about 900 mg to about 1800 mg, from about 900 mg to about 1700 mg, from about 900 mg to about 1600 mg, from about 900 mg to about 1500 mg, from about 900 mg to about 1400 mg, from about 900 mg to about 1300 mg, from about 900 mg to about 1200 mg, from about 900 mg to about 1100 mg, from about 900 mg to about 1000 mg, from about 1000 mg to about 2500 mg, from about 1000 mg to about 2400 mg, from about 1000 mg to about 2300 mg, from about 1000 mg to about 2200 mg, from about 1000 mg to about 2100 mg, from about 1000 mg to about 1900 mg, from about 1000 mg to about 2000 mg, from about 1000 mg to about 1800 mg, from about 1000 mg to about 1700 mg, from about 1000 mg to about 1600 mg, from about 1000 mg to about 1500 mg, from about 1000 mg to about 1400 mg, from about 1000 mg to about 1300 mg, from about 1000 mg to about 1200 mg, from about 1000 mg to about 1100 mg, from about 1100 mg to about 2500 mg, from about 1100 mg to about 2400 mg, from about 1100 mg to about 2300 mg, from about 1100 mg to about 2200 mg, from about 1100 mg to about 2100 mg, from about 1100 mg to about 1900 mg, from about 1100 mg to about 2000 mg, from about 1100 mg to about 1800 mg, from about 1100 mg to about 1700 mg, from about 1100 mg to about 1600 mg, from about 1100 mg to about 1500 mg, from about 1100 mg to about 1400 mg, from about 1100 mg to about 1300 mg, from about 1100 mg to about 1200 mg, from about 1200 mg to about 2500 mg, from about 1200 mg to about 2400 mg, from about 1200 mg to about 2300 mg, from about 1200 mg to about 2200 mg, from about 1200 mg to about 2100 mg, from about 1200 mg to about 1900 mg, from about 1200 mg to about 2000 mg, from about 1200 mg to about 1800 mg, from about 1200 mg to about 1700 mg, from about 1200 mg to about 1600 mg, from about 1200 mg to about 1500 mg, from about 1200 mg to about 1400 mg, from about 1200 mg to about 1300 mg, from about 1300 mg to about 2500 mg, from about 1300 mg to about 2400 mg, from about 1300 mg to about 2300 mg, from about 1300 mg to about 2200 mg, from about 1300 mg to about 2100 mg, from about 1300 mg to about 1900 mg, from about 1300 mg to about 2000 mg, from about 1300 mg to about 1800 mg, from about 1300 mg to about 1700 mg, from about 1300 mg to about 1600 mg, from about 1300 mg to about 1500 mg, from about 1300 mg to about 1400 mg, from about 1400 mg to about 2500 mg, from about 1400 mg to about 2400 mg, from about 1400 mg to about 2300 mg, from about 1400 mg to about 2200 mg, from about 1400 mg to about 2100 mg, from about 1400 mg to about 1900 mg, from about 1400 mg to about 2000 mg, from about 1400 mg to about 1800 mg, from about 1400 mg to about 1700 mg, from about 1400 mg to about 1600 mg, from about 1400 mg to about 1500 mg, from about 1500 mg to about 2500 mg, from about 1500 mg to about 2400 mg, from about 1500 mg to about 2300 mg, from about 1500 mg to about 2200 mg, from about 1500 mg to about 2100 mg, from about 1500 mg to about 1900 mg, from about 1500 mg to about 2000 mg, from about 1500 mg to about 1800 mg, from about 1500 mg to about 1700 mg, from about 1500 mg to about 1600 mg, from about 1600 mg to about 2500 mg, from about 1600 mg to about 2400 mg, from about 1600 mg to about 2300 mg, from about 1600 mg to about 2200 mg, from about 1600 mg to about 2100 mg, from about 1600 mg to about 1900 mg, from about 1600 mg to about 2000 mg, from about 1600 mg to about 1800 mg, from about 1600 mg to about 1700 mg, from about 1700 mg to about 2500 mg, from about 1700 mg to about 2400 mg, from about 1700 mg to about 2300 mg, from about 1700 mg to about 2200 mg, from about 1700 mg to about 2100 mg, from about 1700 mg to about 1900 mg, from about 1700 mg to about 2000 mg, from about 1700 mg to about 1800 mg, from about 1800 mg to about 2500 mg, from about 1800 mg to about 2400 mg, from about 1800 mg to about 2300 mg, from about 1800 mg to about 2200 mg, from about 1800 mg to about 2100 mg, from about 1800 mg to about 1900 mg, from about 1800 mg to about 2000 mg, from about 1000 mg to about 1900 mg, from about 2000 mg to about 2500 mg, from about 2000 mg to about 2400 mg, from about 2000 mg to about 2300 mg, from about 2000 mg to about 2200 mg, from about 2000 mg to about 2100 mg, from abou 250 mg to about 750 mg, from about 250 mg to about 500 mg, from about 300 mg to about 800 mg, or from about 350 mg to about 400 mg of the composition described herein.

In another aspect, the present disclosure provides a method for improving glycemic control or normalizing glucose levels in a subject in need thereof comprising administering to the subject an effective amount of the composition, nutritional supplement or dosage form described herein. The present disclosure also provides the use of the composition, nutritional supplement or dosage form described herein for improving glycemic control or normalizing glucose levels in a subject. The present disclosure also provides the use of the composition or dosage form described herein for the manufacture of a medicament or nutritional supplement for improving glycemic control in a subject.

The present disclosure also provides the composition, nutritional supplement or dosage form described herein for improving glycemic control or normalizing glucose levels in a subject.

In an embodiment, the improvement of glycemic control comprises reducing or lowering hemoglobin A1c (HbA1c) level. In another embodiment, the improvement of glycemic control comprises reducing or lowering fasting glucose levels. In another embodiment, the improvement of glycemic control comprises reducing or lowering post-prandial glucose levels. In another embodiment, the improvement of glycemic control comprises reducing or lowering post-prandial glycemia.

In another aspect, the present disclosure provides a method for lowering hemoglobin A1c (HbA1c) level in a subject in need thereof comprising administering to the subject an effective amount of the composition, nutritional supplement or dosage form described herein. The present disclosure also provides the use of the composition, nutritional supplement or dosage form described herein for lowering HbA1c level in a subject. The present disclosure also provides the use of the composition described herein for the manufacture of a medicament or nutritional supplement for lowering HbA1c level in a subject. The present disclosure also provides the composition, nutritional supplement or dosage form described herein for lowering HbA1c level in a subject.

In embodiments, the HbA1c level is lowered by an amount greater than 0.25% relative to that of prior to the treatment. In embodiments, the HbA1c level is lowered by an amount greater than 0.3% relative to that of prior to the treatment. In embodiments, the HbA1c level is lowered by an amount greater than 0.4% relative to that of prior to the treatment. In embodiments, the HbA1c level is lowered by an amount greater than 0.1% relative to that of prior to the treatment. In embodiments, the HbA1c level is lowered by an amount greater than 0.2% relative to that of prior to the treatment. In embodiments, the HbA1c level is lowered by an amount greater than 0.5% relative to that of prior to the treatment. In embodiments, the HbA1c level is lowered by an amount greater than 0.6% relative to that of prior to the treatment. In embodiments, the HbA1c level is lowered by an amount greater than 0.7% relative to that of prior to the treatment. In embodiments, the HbA1c level is lowered by an amount greater than 0.75% relative to that of prior to the treatment.

In an embodiment, the subject has an HbA1c level of 5.5% or more prior to treatment. In an embodiment, the subject has an HbA1c level of 6.5% or more prior to treatment. In an embodiment, the subject has an HbA1c level of 6.0% or more prior to treatment. In an embodiment, the subject has an HbA1c level of 7.0% or more prior to treatment. In an embodiment, the subject has an HbA1c level of 7.5% or more prior to treatment. In an embodiment, the subject has an HbA1c level of 8.0% or more prior to treatment. In an embodiment, the subject has an HbA1c level of 8.5% or more prior to treatment. In an embodiment, the subject has an HbA1c level of 9.0% or more prior to treatment. In an embodiment, the subject has an HbA1c level of 9.5% or more prior to treatment.

In an embodiment, the method or use also reduces fasting glycemic levels in the subject. In an embodiment, the method or use also reduces the subject's body weight. In an embodiment, the method or use also causes weight loss. In an embodiment, the method or use also reduces the subject's weight as compared to the subject's weight prior to the treatment.

In an embodiment, the subject has a body mass index (BMI) of 18.5 kg/m² or more. In an embodiment, the subject has a body mass index (BMI) of 19.0 kg/m² or more. In an embodiment, the subject has a body mass index (BMI) of 19.5 kg/m² or more. In an embodiment, the subject has a body mass index (BMI) of 20 kg/m² or more. In an embodiment, the subject has a body mass index (BMI) of 20.5 kg/m² or more. In an embodiment, the subject has a body mass index (BMI) of 2 kg/m² or more. In an embodiment, the subject has a body mass index (BMI) of 21.5 kg/m² or more. In an embodiment, the subject has a body mass index (BMI) of 22 kg/m² or more. In an embodiment, the subject has a body mass index (BMI) of 22.5 kg/m² or more. In an embodiment, the subject has a body mass index (BMI) of 23 kg/m² or more. In an embodiment, the subject has a body mass index (BMI) of 23.5 kg/m² or more. In an embodiment, the subject has a body mass index (BMI) of 24 kg/m² or more. In an embodiment, the subject has a body mass index (BMI) of 24.5 kg/m² or more. In an embodiment, the subject has a body mass index (BMI) of 25 kg/m² or more. In an embodiment, the subject has a body mass index (BMI) of 25.5 kg/m² or more. In an embodiment, the subject has a body mass index (BMI) of 26 kg/m² or more. In an embodiment, the subject has a body mass index (BMI) of 26.5 kg/m² or more. In an embodiment, the subject has a body mass index (BMI) of 27 kg/m² or more. In an embodiment, the subject has a body mass index (BMI) of 27.5 kg/m² or more. In an embodiment, the subject has a body mass index (BMI) of 28 kg/m² or more. In an embodiment, the subject has a body mass index (BMI) of 28.5 kg/m² or more. In an embodiment, the subject has a body mass index (BMI) of 29 kg/m² or more. In an embodiment, the subject has a body mass index (BMI) of 29.5 kg/m² or more. In an embodiment, the subject has a body mass index (BMI) of 30 kg/m² or more. In an embodiment, the subject has a body mass index (BMI) of 35 kg/m² or more. In another embodiment, the subject has a BMI of 18.5 to 25 kg/m². In another embodiment, the subject has a BMI of 25 to 30 kg/m². In another embodiment, the subject has a BMI of 25 to 35 kg/m². In another embodiment, the subject has a BMI of 25 to 30 Kg/m². In another embodiment, the subject has a BMI of 35 to 40 kg/m². In another embodiment, the subject has a BMI of 18.5 to 40 kg/m².

In an embodiment, the subject suffers from a metabolic disease. In an embodiment, the subject suffers from metabolic syndrome.

In another aspect, the present disclosure provides a method for treating a metabolic disease in a subject in need thereof comprising administering to the subject an effective amount of the composition, nutritional supplement or dosage form described herein. The present disclosure also provides the use of the composition, nutritional supplement or dosage form described herein for treating a metabolic disease in a subject. The present disclosure also provides the use of the composition described herein for the manufacture of a medicament or nutritional supplement for treating a metabolic disease in a subject. The present disclosure also provides the composition, nutritional supplement or dosage form described herein for use in the treatment of a metabolic disease in a subject.

In another aspect, the present disclosure provides a method for treating a metabolic syndrome in a subject in need thereof comprising administering to the subject an effective amount of the composition, nutritional supplement or dosage form described herein. The present disclosure also provides the use of the composition, nutritional supplement or dosage form described herein for treating a metabolic syndrome in a subject. The present disclosure also provides the use of the composition described herein for the manufacture of a medicament or nutritional supplement for treating a metabolic syndrome in a subject. The present disclosure also provides the composition, nutritional supplement or dosage form described herein for use in the treatment of a metabolic syndrome in a subject.

In embodiments, the metabolic disease is pre-diabetes or diabetes. In embodiments, the metabolic disease is a metabolic syndrome. In embodiments, the metabolic disease is glucose intolerance. In embodiments, the diabetes is type 2 diabetes.

In an embodiment, the treatment is for at least 1 month. In an embodiment, the treatment is for at least 2 months. In another embodiment, the treatment is for at least 1 to 3 months. In another embodiment, the treatment is for at least 2 or 3 months.

Any suitable amount of the composition, nutritional supplement or dosage form may be administered to a subject. The dosages will depend on many factors including the mode of administration. Typically, the amount of the composition contained within a single dose will be an amount that effectively improves glycemic control or normalizes glucose levels in a subject without inducing significant toxicity.

The composition, nutritional supplement or dosage form described herein may be administered according to any suitable dosage regimen. In an embodiment, the composition, nutritional supplement or dosage form is administered or is for administration twice-a-day. In an embodiment, the composition, nutritional supplement or dosage form is administered or is for administration once-a-day. In an embodiment, the composition, nutritional supplement or dosage form is administered or is for administration once every two days. In an embodiment, the composition, nutritional supplement or dosage form is administered or is for administration once every three days. In an embodiment, the composition, nutritional supplement or dosage form is administered or is for administration twice-a-week. In an embodiment, the composition, nutritional supplement or dosage form is administered or is for administration once-a-week.

In an embodiment, the above-mentioned treatment comprises the use/administration of the composition, nutritional supplement or dosage form described herein, in combination with one or more additional drugs, e.g., an anti-diabetic drug such as a hypoglycemic agent. In embodiments, the composition, nutritional supplement or dosage form may be administered in combination with one or more hypoglycemic agents such as a biguanide, a dipeptidyl peptidase-4 (DPP-4) inhibitor, a sulphonylurea, a thiazolidinedione, a meglitinide (glinide), an alpha-glucosidase blocker, a glucagon-like peptide-1 receptor agonist, a sodium-glucose cotransporter-2 (SGLT-2) inhibitor, insulin or an insulin analog.

In an embodiment, the additional anti-diabetic drug is formulated in the same dosage form as the composition or nutritional supplement described herein. In another embodiment, the additional anti-diabetic drug is formulated in a different dosage form. When the composition and the additional anti-diabetic drug are contained in different dosage forms, they may be co-administered simultaneously or separately. Co-administration in the context of the present disclosure refers to the administration of more than one therapeutic in the course of a coordinated treatment to achieve an improved clinical outcome. Such co-administration may also be coextensive, that is, occurring during overlapping periods of time. For example, a first agent may be administered to a patient before, concomitantly, before and after, or after a second active agent is administered.

### EXAMPLES

The present disclosure is illustrated in further details by the following non-limiting examples.

### Example 1: Preparation of Dosage Forms

Capsules comprising the following ingredients were prepared

| Ingredients | Dosing per capsule | | Daily dosing | |
|---|---|---|---|---|
| Chromium (Chromium picolonate) | 250 | μg | 500 | μg |
| Zinc (zinc oxide) | 40 | mg | 80 | mg |
| Copper (copper citrate) | 1 | mg | 2 | mg |
| Magnesium (Magnesium Sulfate) | 30 | mg | 60 | mg |
| Bitter Melon extract | 10 | mg | 20 | mg |
| Fenugreek extract | 10 | mg | 20 | mg |
| Cassia Cinnamon extract | 100 | mg | 200 | mg |
| Thiamine | 50 | mg | 100 | mg |
| Ginger extract | 50 | mg | 100 | mg |
| Coenzyme Q10 | 50 | mg | 100 | mg |
| American Ginseng extract | 10 | mg | 20 | mg |

In embodiments, the capsules are administered twice-a-day to subjects who would benefit from improvement in glucose control.

### Example 2: Efficacy and Safety of the Dosage Forms of Example 1 in Type 2 Diabetes Management: A Double-Blind, Randomised, Placebo Controlled, Parallel-Group, Multicenter Trial

### Methodology

Double-blind randomised placebo-controlled trial to evaluate the effects of daily administration of two capsules according to Example 1 in subjects with type 2 diabetes. The primary outcome was to evaluate the effect of the treatment on HbA1c and the secondary outcomes included safety profile, effect on the cholesterol profile, liver profile, blood pressure and body weight.

### Inclusion Criteria

18 years of age
BMI: <40 Kg/m²
Diabetes type 2 on
All medications allowed
A1c≤10%

### Exclusion Criteria

Type 1 Diabetes
BMI: ≤18.5 or ≥40 Kg/m²
eGFR≤30 ml/min/1.73 m²
Moderate-severe liver failure

### Study Design

Patients were administered the treatment (2 capsules/day) or placebo for a period of 3 months;
Fasting glucose, A1c, Creatine, GFR, Total Cholesterol, LDL cholesterol, C-reactive protein and AST/ALT were assessed at day 0 and at the end of the study (3 months);
Anthopometric evolution (Height, Weight, Blood Pressure) was assessed at day 0 and at the end of the study (3 months);
Prandial glycemic and post-prandial glycemic were tested at day 0, 1 month, 2 months and 3 months

### Study Population

The baseline characteristics of the patients who completed the 12-week trial are depicted in

### Table 1.

TABLE 1

| | Treatment | Placebo |
|---|---|---|
| # of participants | 32 | 28 |
| Male/Female | 17/15 | 17/11 |
| Average age (years) | 67.9 | 66.6 |
| Average weight (lbs) | 169.5 | 183.4 |
| Average Body Mass Index, BMI (kg/m²) | 27.6 | 28.2 |
| Average Diabetes duration (years) | 3.3 | 3.1 |
| Average Hemoglobin A1c (%)/mmol/mol | 7.1/54.1 | 6.84/51.3 |

### Results

### A) Glycemia and Weight

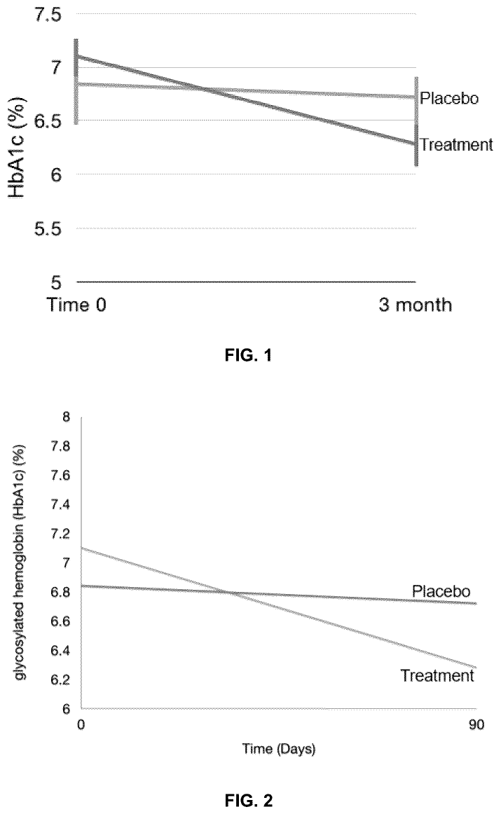

As shown in FIG. 1 and Table 2, the treatment, but not the placebo, led to a significant reduction of HbA1c after 3 months. A trend toward a reduction of the fasting glycemic and body weight was also measured in the treatment group, but not in the placebo group.

TABLE 2

| | Treatment | Placebo |
|---|---|---|
| Δ Hemoglobin A1c (%) | −0.82* | −0.12** |
| Δ weight (lbs) | −0.90 | −0.12 |
| Δ fasting glycemic | −1.84 | 0.21 |

Results subtracted from baseline value at week 12
*P = 0.0131;
**P = 0.6151

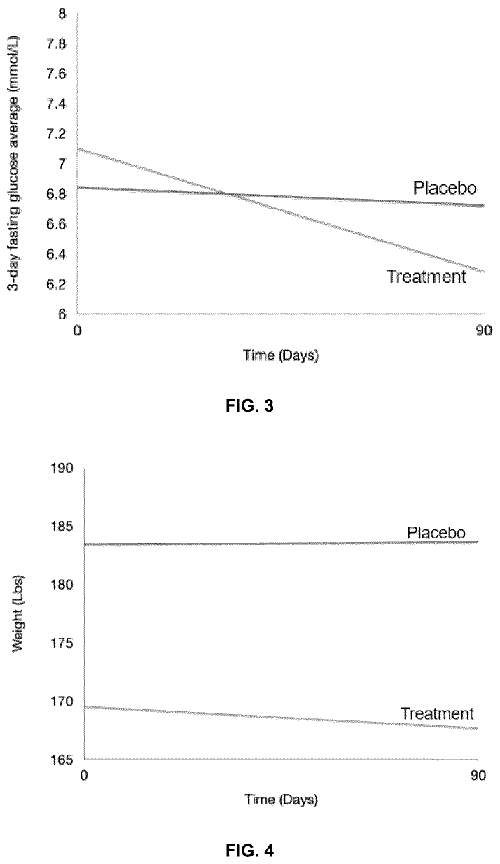

At 12 weeks, the treatment was associated with a 11.55% HbA1c decrease (7.10 to 6.28%) and a 11.62% decrease in 3-day fasting glucose average (7.92 to 7.00 mmol/L) while the placebo group had a 1.75% HbA1c decrease (6.84 to 6.72%) and a 2.46% 3-day fasting glucose average decrease (7.7 to 7.51 mmol/L). In regards to the weight, the treatment was associated with 1.08% weight loss [(169.5 to 167.66 lbs) or (76.88 to 76.05 kg)] while the placebo was associated with a 0.12% weight gain [(183.39 to 183.61 lbs) or (83.18 to 83.28 kg)] (Table 3; FIGS. 2-4).

The results confirms the efficacy of the treatment in glycemic management with a clinically significant HbA1c and 3-day fasting glycemia effect of at least a 10% reduction. This analysis also suggests that the treatment is not associated with weight gain.

TABLE 3

| | | |
|---|---|---|
| | | Difference between |
| Beginning of Trial | End of Trial | beginning and end |
| (Day 0) | (Day 90) | of trial (%) |

3-day fasting glucose average (mmol/L)

| | | | |
|---|---|---|---|
| Placebo | 7.7 | 7.51 | −2.46 |
| Treatment | 7.92 | 7.00 | −11.62 |

Weight (lbs)

| | | | |
|---|---|---|---|
| Placebo | 183.39 | 183.61 | 0.12 |
| Treatment | 169.5 | 167.66 | −1.08 |

Glycosylated hemoglobin (HbA1c)

| | | | |
|---|---|---|---|
| Placebo | 6.84 | 6.72 | −1.75 |
| Treatment | 7.10 | 6.28 | −11.55 |

### B) Adverse Events

Adverse events reported by study participants are depicted in Table 4. The most frequent adverse events in the treatment group were mild and transient gastrointestinal events including nausea (9.3% vs. 3.5% in placebo group) and dyspepsia (15.6% vs. 10.7% in placebo group).

TABLE 4

| | Treatment | Placebo |
|---|---|---|
| Any adverse events (n) | 10 | 4 |
| Nausea (n/%) | 3/9% | 1/3% |
| Dyspepsia (n/%) | 5/15% | 3/10% |
| Hypoglycemia (n/%) | 2* (6%) | 0 |

*The two participants reported minor hypoglycemic events; both patients were using long-active insulin analogues and no further events occurred after a 5% decrease of their insulin.

### C) Lipid Profile

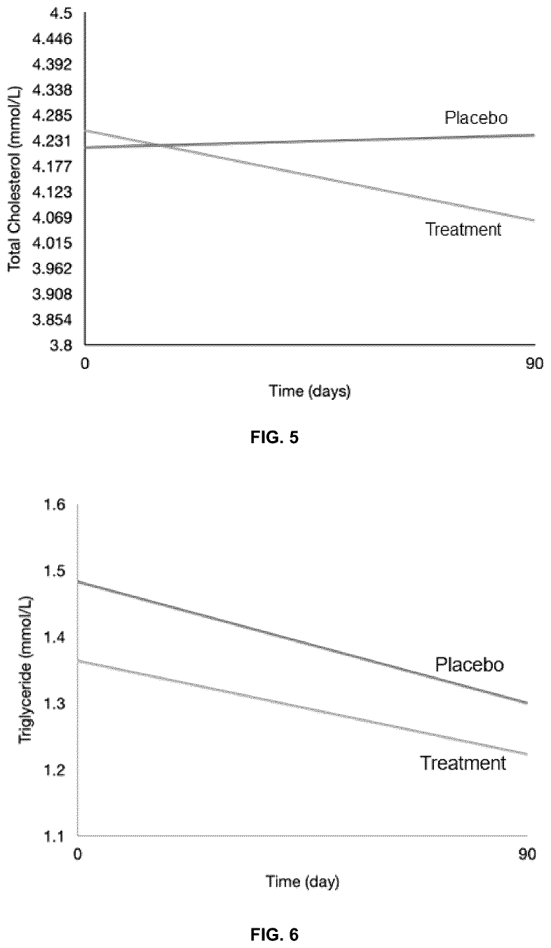

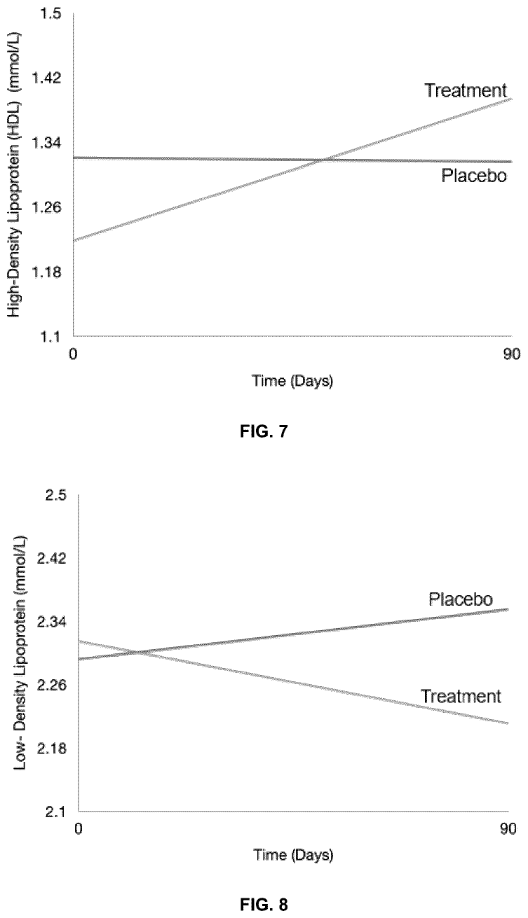

At 12 weeks, treatment with the formulation according to Example 1 decreased total cholesterol by 4.47% while placebo increased it by 0.62%. The active arm had a decrease in triglycerides of 10.33% while the placebo arm had a triglyceride lowering of 12.44%. The treatment further improved the lipid profile by reducing LDL by 7.45% while placebo increased it by 2.70%. Moreover, the treatment increased HDL by 14.52% while placebo reduced it by 0.38% (Table 5; FIGS. 5-8).

Treatment with the formulation according to Example led to a favorable effect regarding the lowering of total cholesterol and LDL cholesterol, and increasing HDL. These results suggest that the formulation could be, in addition to its's proven glucose lowering abilities, an alternative and/or an addition to type 2 diabetes management in people who are reluctant to initiate or add new antidiabetic agents while also providing them with some lipid profile benefits.

TABLE 5

| | | |
|---|---|---|
| Beginning of Trial | End of Trial | Difference between beginning |
| (Day 0) | (Day 90) | and end of trial (%) |

Total Cholesterol (mmol/L)

| | | | |
|---|---|---|---|
| Placebo | 4.217 | 4.243 | 0.62 |
| Treatment | 4.252 | 4.062 | −4.47 |

Triglyceride (mmol/L)

| | | | |
|---|---|---|---|
| Placebo | 1.484 | 1.300 | −12.40 |
| Treatment | 1.365 | 1.224 | −10.33 |

High-Density Cholesterol (mmol/L)

| | | | |
|---|---|---|---|
| Placebo | 1.321 | 1.316 | −0.38 |
| Treatment | 1.219 | 1.396 | 14.52 |

Low-Density Cholesterol (mmol/L)

| | | | |
|---|---|---|---|
| Placebo | 2.293 | 2.355 | 2.70 |
| Treatment | 2.390 | 2.212 | −7.45 |

### D) Liver Enzymes

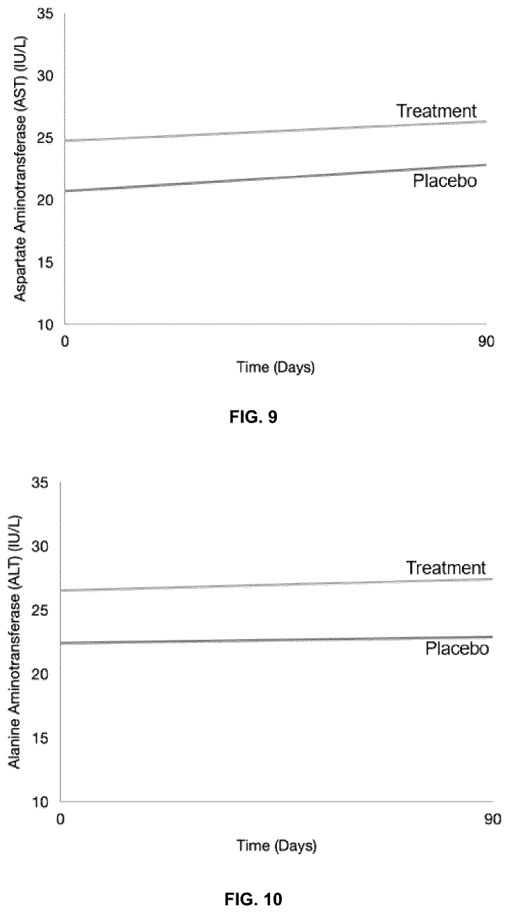

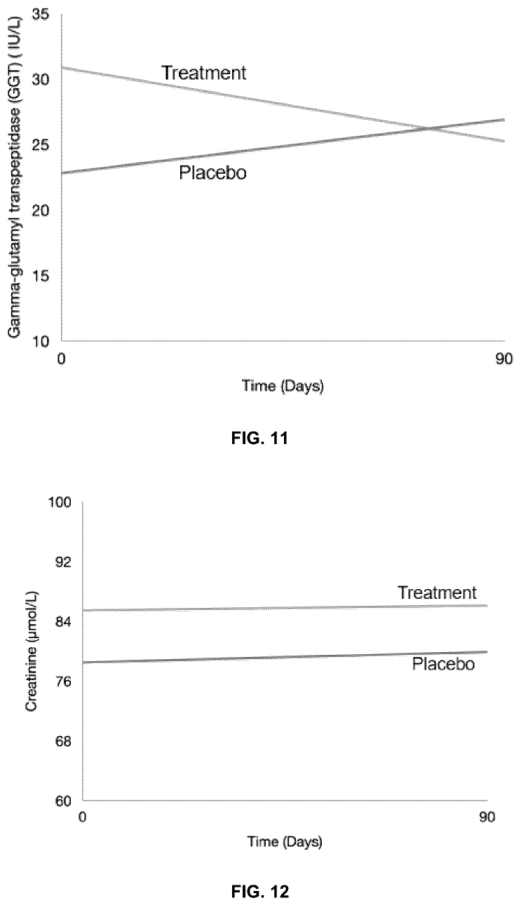

At 12 weeks, treatment with the formulation according to Example 1 was associated with an increase in Aspartate Aminotransferase (AST) and Alanine Aminotransferase (ALT) of 6.35% and 3.39% respectively, while the placebo group had an increase of 10.24% and 2.05% of their AST and ALT respectively. In regards to Gamma-Glutamyl Transpeptidase (GGT), the treatment group had a 18.26% reduction while the placebo had 17.97% reduction (Table 6; FIGS. 9-11).

These suggest that treatment with the formulation according to Example 1 has no effects on liver transpeptidase and transaminase levels.

TABLE 6

| | | |
|---|---|---|
| Beginning of Trial | End of Trial | Difference between beginning |
| (Day 0) | (Day 90) | and end of trial (%) |

Aspartate Aminotransferase (AST) (IU/L)

| | | | |
|---|---|---|---|
| Placebo | 20.69 | 22.81 | 10.24 |
| Treatment | 24.73 | 26.30 | 6.35 |

Alanine Aminotransferase (ALT) (IU/L)

| | | | |
|---|---|---|---|
| Placebo | 22.42 | 22.88 | 2.05 |
| Treatment | 26.53 | 27.43 | 3.39 |

Gamma-Glutamyl Transpeptidase (GGT) (IU/L)

| | | | |
|---|---|---|---|
| Placebo | 22.81 | 26.91 | 17.97 |
| Treatment | 30.89 | 25.25 | −18.26 |

### E) Renal Safety

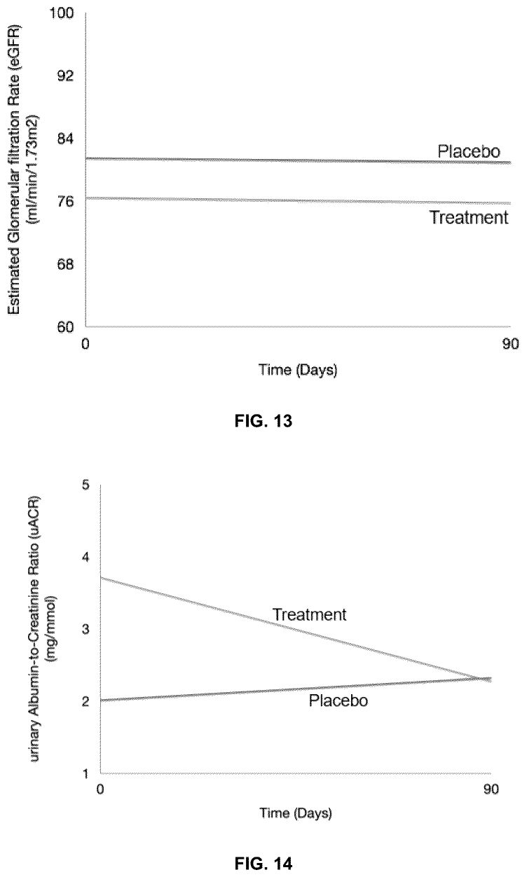

At 12 weeks, treatment with the formulation according to Example 1 was associated with a 0.77% creatinine increase (85.47 to 86.13 μmol/L) and a 0.85% eGFR decrease (76.38 to 75.73 ml/min/1.73 m²) while the placebo group had a 1.77% creatinine increase (78.5 to 79.89 μmol/L) and a 0.65% eGFR decrease (81.4 to 80.8 ml/min/1.73m²). In regards to the urinary Albumin-Creatinine Ratio, the treatment was associated with a 38% reduction (3.70 to 2.267 mg/mmol) while the placebo group had a 15.26% increase (2.01 to 2.32 mg/mmol) (Table 7; FIGS. 12-14).

The results of this analysis suggest that the treatment is safe regarding renal function while improving albuminuria.

TABLE 7

| | | |
|---|---|---|
| Beginning of Trial | End of Trial | Difference between beginning |
| (Day 0) | (Day 90) | and end of trial (%) |

Creatinine (μmol/L)

| | | | |
|---|---|---|---|
| Placebo | 78.5 | 79.89 | 1.77 |
| Treatment | 85.47 | 86.13 | 0.77 |

Estimated Glomerular filtration Rate (eGFR) (ml/min/1.73 m²)

| | | | |
|---|---|---|---|
| Placebo | 81.43 | 80.89 | −0.66 |
| Treatment | 76.38 | 75.73 | −0.85 |

Urinary Albumin-to-Creatinine Ratio (uACR) (mg/mmol)

| | | | |
|---|---|---|---|
| Placebo | 2.01 | 2.32 | 15.42 |
| Treatment | 3.71 | 2.27 | −38.81 |

### Example 3: Preparation of Dosage Forms

Capsules comprising the following ingredients were prepared

TABLE 8

| | Dosing | | Daily | |
|---|---|---|---|---|
| Ingredients | per capsule | | dosing | |
| Chromium | 250 | μg | 500 | μg |
| (Chromium picolonate) | | | | |
| Zinc (zinc oxide) | 40 | mg | 80 | mg |
| Copper (copper citrate) | 1 | mg | 2 | mg |
| Magnesium | 150 | mg | 300 | mg |
| (Magnesium Sulfate) | | | | |
| Bitter Melon extract | 10 | mg | 20 | mg |
| Fenugreek extract | 10 | mg | 20 | mg |
| Cassia Cinnamon extract | 100 | mg | 200 | mg |
| Thiamine | 50 | mg | 100 | mg |
| Ginger extract | 50 | mg | 100 | mg |
| Coenzyme Q10 | 50 | mg | 100 | mg |
| American Ginseng extract | 10 | mg | 20 | mg |

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise.

What is claimed is:

1. A composition comprising at least five of the following ingredients:
(a) coenzyme Q10;
(b) chromium or any pharmaceutically acceptable salt thereof;
(c) zinc or any pharmaceutically acceptable salt thereof;
(d) magnesium or any pharmaceutically acceptable salt thereof;
(e) bitter melon extract;
(f) fenugreek extract;
(g) cinnamon extract;
(h) thiamine;
(i) ginger extract;
(j) ginseng extract; and
(k) copper or any pharmaceutically acceptable salt thereof.

2. The composition of claim 1, which comprises at least 6 of ingredients (a)-(k).

3. The composition of claim 1, which comprises at least 7 of ingredients (a)-(k).

4. The composition of claim 1, which comprises at least 8 of ingredients (a)-(k).

5. The composition of claim 1, which comprises at least 9 of ingredients (a)-(k).

6. The composition of claim 1, which comprises at least 10 of ingredients (a)-(k).

7. The composition of claim 1, which comprises ingredients (a)-(k).

8. The composition of any one of claims 1 to 7, wherein coenzyme Q10 is present in an amount of about 5% to about 25% (w/w).

9. The composition of any one of claims 1 to 8, wherein chromium is present in an amount of about 0.01% to about 5.0% (w/w).

10. The composition of any one of claims 1 to 9, wherein zinc is present in an amount of about 5% to about 15% (w/w).

11. The composition of any one of claims 1 to 10, wherein magnesium is present in an amount of about 4% to about 15% (w/w).

12. The composition of any one of claims 1 to 11, wherein the bitter melon extract is present in an amount of about 1% to about 15% (w/w).

13. The composition of any one of claims 1 to 12, wherein the fenugreek extract is present in an amount of about 1% to about 15% (w/w).

14. The composition of any one of claims 1 to 13, wherein the cinnamon extract is present in an amount of about 20% to about 70% (w/w).

15. The composition of any one of claims 1 to 14, wherein thiamine is present in an amount of about 10% to about 25% (w/w).

16. The composition of any one of claims 1 to 15, wherein the ginger extract is present in an amount of about 10% to about 30% (w/w).

17. The composition of any one of claims 1 to 16, wherein the ginseng extract is present in an amount of about 1% to about 20% (w/w).

18. The composition of any one of claims 1 to 17, wherein copper is present in an amount of about 0.1% to about 5.0% (w/w).

19. The composition of any one of claims 1 to 18, wherein the composition comprises about 30 mg to about 120 mg of coenzyme Q10.

20. The composition of any one of claims 1 to 19, wherein the composition comprises about 0.1 mg to about 0.8 mg of chromium.

21. The composition of any one of claims 1 to 20, wherein the composition comprises about 20 mg to about 100 mg of zinc.

22. The composition of any one of claims 1 to 21, wherein the composition comprises about 10 mg to about 500 mg of magnesium.

23. The composition of any one of claims 1 to 22, wherein the composition comprises about 5 mg to about 50 mg of the bitter melon extract.

24. The composition of any one of claims 1 to 23, wherein the composition comprises about 5 mg to about 50 mg of the fenugreek extract.

25. The composition of any one of claims 1 to 24, wherein the composition comprises about 50 mg to about 500 mg of the cinnamon extract.

26. The composition of any one of claims 1 to 25, wherein the composition comprises about 30 mg to about 150 mg of thiamine.

27. The composition of any one of claims 1 to 26, wherein the composition comprises about 25 mg to about 125 mg of the ginger.

28. The composition of any one of claims 1 to 27, wherein the composition comprises about 5 mg to about 250 mg of the ginseng extract.

29. The composition of any one of claims 1 to 28, wherein the composition comprises about 0.5 mg to about 5.0 mg of copper.

30. The composition of any one of claims 1 to 29, wherein the composition further comprises one or more pharmaceutically or nutritionally acceptable excipients.

31. A dosage form or nutritional supplement comprising the composition of any one of claims 1 to 30.

32. The dosage form or nutritional supplement of claim 31, which is a capsule.

33. The composition of any one of claims 1 to 30 or the dosage form or nutritional supplement of claim 31 or 32, for use in at least one of normalizing glucose levels, normalizing dysglycemia, improving fasting glucose, improving HbA1c, improving blood sugar, improving blood glucose, or improving glycemic control in a subject.

34. The composition, dosage form or nutritional supplement for use according to claim 33, wherein the subject suffers from a metabolic condition or metabolic disease.

35. The composition, dosage form or nutritional supplement for use according to claim 34, wherein the metabolic disease is a metabolic syndrome, prediabetes, or Type II diabetes.

36. The composition, dosage form or nutritional supplement for use according to claim 34, wherein the metabolic condition is a metabolic syndrome, prediabetes, or Type II diabetes.

37. The composition, dosage form or nutritional supplement for use according to any one of claims 33 to 35, wherein the dosage form or nutritional supplement is for twice-a-day administration.

38. Use of the composition of any one of claims 1 to 30 or the dosage form or nutritional supplement of claim 31 or 32, for the preparation of a medicament for normalizing glucose levels, normalizing dysglycemia, improving fasting glucose, improving HbA1c, improving blood sugar, improving blood glucose or improving glycemic control in a subject.

39. The use according to claim 38, wherein the subject suffers from a metabolic condition or a metabolic disease.

40. The use according to claim 39, wherein the metabolic condition is metabolic syndrome, prediabetes, or Type II diabetes.

41. The use according to any one of claims 38 to 40, wherein the medicament or nutritional supplement is for twice-a-day administration.

42. A method for normalizing glucose levels, normalizing dysglycemia, improving fasting glucose, improving HbA1c, improving blood sugar, improving blood glucose, or improving glycemic control in a subject comprising administering to the subject an effective amount of the composition of any one of claims 1 to 30 or the dosage form or nutritional supplement of claim 31 or 32.

43. The method of claim 42, wherein the subject suffers from a metabolic condition or a metabolic disease.

44. The method of claim 43, wherein the metabolic condition is metabolic syndrome, prediabetes, or Type II diabetes.

45. The method of any one of claims 42 to 44, wherein the dosage form is administered twice-a-day.
